SEQUENCE LISTING (4 sequences)

SEQ ID NO: 1 - DNA, 20 bp, Artificial Sequence
  Forward Primer as set forth in Example 1
cccctgtgaggaactactgt

SEQ ID NO: 2 - DNA, 19 bp, Artificial Sequence
  Reverse primer as set forth in Example 1
gaccactatggctctcccg

SEQ ID NO: 3 - DNA, 25 bp, Artificial Sequence
  Atto633-conjugated probe as set forth in Example 1
agccatggcgttagtatgagtgtcg

SEQ ID NO: 4 - DNA, 18 bp, Artificial Sequence
  Reverse primer as set forth in Example 2
accactatggctctcccg

(19) United States
(12) Patent Application Publication
(10) Pub. No.: US 2020/0063189 A1
(43) Pub. Date: Feb. 27, 2020

(54) SYSTEM AND METHOD FOR PURIFYING AND AMPLIFYING NUCLEIC ACIDS

(71) Applicant: miDiagnostics NV, Leuven (BE)

(72) Inventors: William OSBURN, Baltimore, MD (US); Maarten FAUVART, Leuven (BE); Rita VOS, Leuven (BE); Rodrigo WIEDERKEHR, Leuven (BE)

(21) Appl. No.: 16/489,111

(22) PCT Filed: Feb. 23, 2018
(86) PCT No.: PCT/US18/19438

§ 371 Date: Aug. 27, 2019

Related U.S. Application Data
(60) Provisional application No. 62/464,097, filed on Feb. 27, 2017.
(60) Provisional application No. 62/554,870, filed on Sep. 6, 2017.

(51) Int. Cl. C12Q 1/6806; C12N 15/10; C12Q 1/70; G01B 15/06; B01L 3/00

(52) U.S. Cl. CPC C12Q 1/6806; C12N 15/1006; G01N 2015/0693; G01B 15/06; B01L 3/502746; C12Q 1/70

(57) ABSTRACT

Provided are compositions, methods, systems, and kits for the purification, or detection, or amplification, or quantitation, of nucleic acids in biological samples. In some embodiments, a single point of care device/reactor is provided.

### CROSS REFERENCE TO RELATED APPLICATIONS

This application claims priority to U.S. Provisional Application No. 62/464,097 titled “SYSTEM AND METHOD FOR PURIFYING AND AMPLIFYING NUCLEIC ACIDS”, filed Feb. 27, 2017, and U.S. Provisional Application No. 62/554,870 titled “SYSTEM AND METHOD FOR PURIFYING AND AMPLIFYING NUCLEIC ACIDS”, filed Sep. 6, 2017, the disclosures of which are herein incorporated by reference in their entirety for all purposes.

### FIELD OF THE DISCLOSURE

The present disclosure relates generally to nucleic acid purification and amplification by the polymerase chain reaction (PCR). More particularly, the present disclosure relates to compositions, systems and methods for performing nucleic acid purification and amplification in a point of care system or device.

### BACKGROUND

Point of care diagnostic systems and methods provide medical professionals with timely diagnostic information without the use of more expensive and time consuming lab-based tests. Typical point of care tests are performed at patient-provider contact, either in a clinician's office or clinic, in a field clinic or field trial, a provider home visit, or similar situation.

As such, many valuable diagnostic tests have been considered impractical for point of care diagnostics. For example, many nucleic acid amplification tests identify specific disease vector nucleic acids to accurately diagnose infections and their causes, but are not practical for point of care diagnostics. To amplify nucleic acids from biological specimens, the nucleic acids need to be purified in order to remove digestive enzymes, inhibitor molecules and other contaminants present in the specimen that would inhibit nucleic acid amplification reactions, such as RT-qPCR and PCR reactions. In addition, these tests are extremely sensitive to environmental contamination, an issue in many point of care settings both within and outside of medical facilities. In short, existing nucleic acid purification and amplification methods are time consuming, may use relatively large volumes of reagents, require separation or centrifugation steps, or utilize expensive microbead or similar substrates that require time consuming recovery and recycling. It would therefore be advantageous to provide devices, compositions and methods of separating, amplifying and qualitatively and/or quantitatively analyzing nucleic acids that could be used in a point of care device. The present disclosure is pertinent to this need.

### SUMMARY

There is a need in the art for nucleic acid extraction, purification and analysis methods and systems that can be implemented with low volumes of reagents and without using beads or centrifugation. This disclosure addresses the foregoing concerns by providing for the purification of nucleic acids in a microfluidic environment. While existing lab methods for nucleic acid purification using silica-based chromatography require reagents and methodology that are not suitable for use on a chip-based, stand-alone diagnostic device, the present disclosure is designed to facilitate extraction, separation, amplification and analysis of nucleic acids on the same device as the subsequent quantification assay.

In one aspect, the present disclosure provides a method for the purification of nucleic acids from a biological sample, including the steps of delivering the unpurified nucleic acids into a microfluidic region, contacting the nucleic acids with a fixed surface in the microfluidic region, wherein the nucleic acids adhere to the surface; washing the microfluidic region and surface with a first buffer; washing the microfluidic region and surface with a second buffer, where the second buffer has a pH that is equal to or higher than the first buffer.

In one aspect, the fixed surface comprises a silicon oxide or metal oxide or nitride. In one aspect, the metal oxide comprises, aluminum oxide, or hafnium oxide.

In one aspect, the present disclosure also provides for amplifying at least some of the nucleic acids to produce an amplification product; and detecting the amplification product. In one aspect, delivering the nucleic acids to the microfluidic region includes adding a biological sample to an adhesion buffer, disrupting cells, viruses or bacteria in the adhesion buffer so that the nucleic acids are mixed with the adhesion buffer. Disruption can include cell/virus lysis. The lysis can be performed using any suitable approaches, which include but are not necessarily limited to thermal, chemical and mechanical based lysis.

In one aspect, the present disclosure provides a method for the purification, amplification, and detection of the presence or absence of a nucleic acid in a sample. In embodiments the disclosure provides for generation and analysis of nucleic acid amplification products, including the steps of delivering the unpurified nucleic acids into a microfluidic region, contacting the nucleic acids with a fixed surface in the microfluidic region, wherein the nucleic acids adhere to the surface; washing the microfluidic region and surface with a first buffer; washing the microfluidic region and surface with a second buffer, where in certain approaches the second buffer has a pH that is equal to or higher than the first buffer; amplifying at least some of the nucleic acids to produce an amplification product(s); and detecting the amplification product(s). In one aspect, delivering the nucleic acids to the microfluidic region includes obtaining a biological sample known to or suspected of comprising or potentially comprising nucleic acids, adding the sample to an adhesion buffer, and lysing or otherwise disrupting cells and/or viruses in the adhesion buffer so that the viral or cellular contents are mixed with the adhesion buffer. In one aspect, the step of delivering the nucleic acids to the microfluidic region uses an adhesion solution comprising kosmotropic salts and a nuclease inhibitor. In embodiments, one or more solutions, including but not limited to buffers, used in embodiments of the disclosure are free of organic solvents. In embodiments the solutions are ethanol free, are chaotropic salt-free, or are both organic solvent and chaotropic salt free. Those skilled in the art will recognize that in certain cases the term “free” may nevertheless comprise trace amounts of chaotropic salts, or organic solvents which include but are not necessarily limited to ethanol or other alcohols that will be apparent to those skilled in the art given the benefit of the present disclosure.

In one aspect of the present disclosure, the purification and amplification steps take place in or on the same device. In one aspect, purification, amplification, and detection all occur in the same device. In one aspect, following detection of the amplification products, the method includes determining the identity of the source of the nucleic acids and reporting this result to the diagnostic provider, creating a record of this result in computer readable media, or both.

### BRIEF DESCRIPTION OF THE DRAWINGS

The accompanying drawings provide visual representations that will be used to more fully describe the representative embodiments disclosed herein and can be used by those skilled in the art to better understand them and their inherent advantages.

FIG. 1 A) Amplification curves and B) Standard curves for the Superscript III-Tfi RT-qPCR performance test.

FIG. 2 A) Amplification curves and B) Standard curves for the Superscript III-AmpTaq360 RT-qPCR performance test.

FIG. 3. Extraction of Hepatitis C virus (HCV) RNA from buffer at different binding pH, elution pH and elution temperatures using a HfO₂ coated surface using a blanket wafer as a non-limiting illustration.

FIG. 4. Titration of KH₂PO₄ concentration in wash buffer during extraction of RNA from plasma using a HfO₂ coated surface.

FIG. 5. Extraction of RNA from 15 different plasma specimens using a HfO₂ coated surface.

FIG. 6. Extraction of RNA from buffer at different binding pH, elution pH and elution temperature using an Al₂O₃ coated surface.

FIG. 7. Titration of NaOAc concentration in wash buffer during extraction of RNA from plasma using an Al₂O₃ coated surface.

FIG. 8. Results from extraction of RNA from 15 different plasma specimens using an Al₂O₃ coated reactor.

FIG. 9. Results of the extraction of RNA using a reaction surface with a pillar structures.

FIG. 10. (A) Data for Cycle threshold (Ct) obtained by extraction and purification of RNA from buffer on scraped pillar surfaces. (B) RNA recovery results.

FIG. 11. RNA recovery results obtained using binding buffers with different pH values.

FIG. 12. RNA recovery results obtained using heated plasma and different salts.

FIG. 13. RNA recovery results obtained using unheated plasma with proteinase.

FIG. 14 depicts data representing extraction of RNA by capillary flow.

FIG. 15. Data showing lysis of viral particles obtained from cell culture and incubated with scraped pillars at different sodium dodecyl sulfate (SDS) concentrations (A) and temperatures (55° C. (B) and 75° C. (C)).

FIG. 16. Data representing fluorescence normalization using an internal control RNA and HCV RNA.

FIG. 17. Scanning electron micrograph (SEM) of representative micropillar configuration.

FIG. 18. Representative schematic of a vessel design.

FIG. 19. SEM of representative vessel design; right panel illustrates detection segments.

FIG. 20. Graphs depicting results obtained by on-chip amplification of HCV RNA.

FIG. 21. Bench scale amplification of viral RNA. Average Ct values for A) HCV, C) HIV, and E) ZIK V RNA standards (1×10⁶-1×10⁰ copies/μL) performed on the LightCycler480. Average normalized fluorescence curves across three B) HCV, D) HIV, and F) ZIKV standard replicates (4×10⁶-4×10⁰ copies/reaction) on the LightCycler480.

FIG. 22. Bench scale amplification of viral DNA. Average Ct values for A) HPV 16 and C) HPV 18 DNA standards (1×10⁶-1×10⁰ copies/μL) performed on the LightCycler480. Average normalized fluorescence curves across three B) HPV 16 and D) HPV 18 standard replicates (4×10⁶-4×10⁰ copies/reaction) on the LightCycler480.

FIG. 23. Silicon microchip design and performance A) Microreactor design; B) Representative pictures of 1.3 μL microreactor before (left) and after (right) amplification, boxes denote the segments used during quantitation of reaction fluorescence, C) Representative microchip mounted on PCB; D) Laboratory set-up for external detection of reaction fluorescence; E) Distribution of Tm across 93 segments, bars represent the total number of segments with the indicated temperature.

FIG. 24. On-chip amplification of viral RNA. Average Ct values for A) HCV, C) HIV, and E) ZIKV RNA standards (1×10⁶-1×10² copies/μL) performed with 50 cycles in silicon microchip microreactor. Average normalized fluorescence amplification curves across three B) HCV, D) HIV, and F) ZIKV standard replicates (4×10⁵-4×10⁰ copies/reaction) in silicon microchip microreactor.

FIG. 25. On-chip amplification of viral DNA. Average Ct values for A) HPV 16 and B) HPV 18 DNA standards (1×10⁶-1×10¹ copies/μL) performed with 50 cycles on chip. Average normalized fluorescence curves across three HPV 16 standard replicates (4×10⁵-4×10⁰ copies/reaction) on chip. C) Average Ct values for HPV 18 DNA standards (1×10⁶-1×10¹ copies/μL) performed with 50 cycles on chip. Average normalized fluorescence curves across three B) HPV 16 and D) HPV 18 standard (4×10⁵-4×10⁰ copies/reaction) replicates on chip.

### DETAILED DESCRIPTION

Unless defined otherwise herein, all technical and scientific terms used in this disclosure have the same meaning as commonly understood by one of ordinary skill in the art to which this disclosure pertains.

Every numerical range given throughout this specification includes its upper and lower values, as well as every narrower numerical range that falls within it, as if such narrower numerical ranges were all expressly written herein.

The present disclosure related generally to compositions, methods and devices for nucleic acid analysis. Embodiments comprise separating nucleic acids from cellular and/or viral non-non-nucleic acid components, and detecting and/or quantifying the nucleic acids.

In more detail, clinical laboratory-based nucleic acid amplification tests (NAT) play an important role in diagnosing viral infections but their laboratory infrastructure requirements and failure to diagnose at the point of need limit their clinical utility in both resource-rich and—limited clinical settings. The development of fast and sensitive point-of-care (POC) viral NAT may overcome these limitations. The scalability of silicon microchip manufacturing combined with advances in silicon microfluidics present an opportunity for development of rapid and sensitive POC NAT on silicon microchips.

Methods of the present disclosure are implemented using devices comprising chips, which may comprise channels, and wherein on-chip nucleic acid separation, amplification and detection/quantification is performed. The disclosure comprises each process step and all combinations of process steps described here, each component and all combinations of devices and the devices themselves as described herein, each reagent and combinations of reagents described herein, and all combinations of the foregoing components. The methods disclosed herein minimize or entirely avoid the use of the organic solvents and other chaotropic agents that are commonly used in nucleic acid extraction. Thus, certain implementations of this disclosure have advantages in that they avoid use of components that are unsuitable for use outside of clinical or laboratory settings, would increase cost of manufacture, and would otherwise require special shipping/handling of a finished point of care device.

Components pertinent to implementing embodiments of this disclosure comprise one or more fixed surfaces (e.g. silicon wafer, silicon pillars), which may be coated with certain compositions that include but are not limited to a silicon oxide, or a metal oxide, a binding/lysing buffer, one or more wash buffers, one or more elution buffers, the latter of which may also function as amplification buffers.

The surface(s) are present or contained within a microfluidic environment having a volume in certain embodiments from 10 microliters to 1500 nanoliters. In embodiments the volume is less than 5 microliters, less than 2 microliters, or between about 500 to 1500 nanoliters. It is considered without being bound to any particular theory that such reaction volumes and surface areas allow for precise control of purification and amplification, for example, by rapid and precise temperature control, and rapid and precise changes in the microfluidic or nanofluidic shell surrounding each individual nucleic acid to be purified and amplified.

The surfaces are in embodiments silicon oxides or metal oxides or nitrides, such as aluminum oxide (Al₂O₃), hafnium oxide (HfO₂), silicon nitride (Si₃N₄), or silicon oxide (SiO₂). The surfaces provide for adherence (e.g., non-covalent) of the nucleic acids, and retention of the nucleic acids at certain temperatures or in certain microfluidic solutions, and efficient release of the nucleic acids at other temperatures or in other microfluidic solutions. The surfaces are made by techniques such as chemical vapor deposition (CVD) techniques on/within microfluidic vessels produced in part using, for example, silicon oxide wafers. A surface where nucleic acid binding, and/or amplification, and/or detection is performed, may be either open (i.e., flat or smooth) space or it may comprise three dimensional features that are within or on a surface, such as pillars, which can improve surface to volume ratio.

In embodiments three dimensional features of a microfluidic vessel, or a portion thereof, or a surface in a microfluidic vessel of this disclosure, are formed using any suitable approach by modifying a substrate, such as a silicon wafer or silicon nitride substrate. In embodiments the substrate is modified at least in part by a process that comprises etching. By “etching” it is meant that layers are removed from a surface of substrate, such as a wafer, during manufacturing. Given the benefit of this disclosure those skilled in the art will be able to adapt any suitable etching or other approaches to produce surfaces that can be used in various embodiments of the present disclosure, as further described below. In certain approaches etching comprises laser etching, liquid phase etching or plasma phase etching. Liquid phase etching can comprise wet etching or anisotropic wet etching. Similarly, plasma etching can be isotropic or anisotropic. In embodiments a silicon wafer is modified for use in various implementations of this disclosure by deep reactive ion etching. Devices, reagents and methods for deep reactive ion etching are known in the art and can be adapted by those skilled in the art, given the benefit of the present disclosure, to produce microfluidic devices and/or components thereof having surface areas that comprise three-dimensional features, including but not necessarily limited to micropillars.

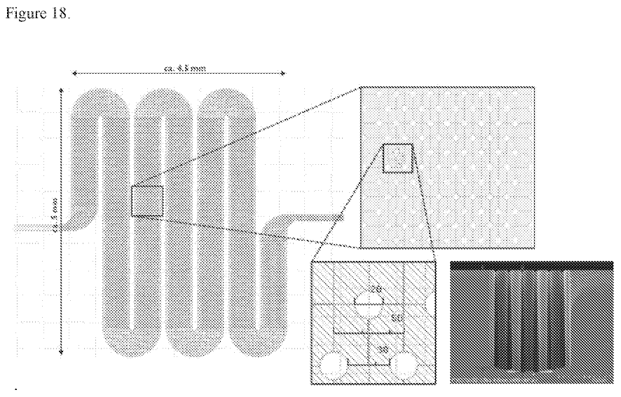

Micropillars comprise dimensions that are suitable for use in the methods and microfluidic components/devices of this disclosure. In non-limiting examples, the micropillars are columnar, and thus may be rectangular or they may have a rounded shape. In embodiments the micropillars are from 190-200 μm in length. The length may be perpendicular relative to a substrate from which the micropillars project, with the understanding that the micropillars may be formed of the same material as the substrate. In certain embodiments the micropillars have a width or diameter of approximately 20 μm. The micropillars are generally configured on a surface such that they provide for adequate flow-through dynamics and surface area whereby a biological sample comprising nucleic acids can be contacted with the micropillar surface area such that at least some of the nucleic acids adhere to the micropillar surface, as further described herein. In certain implementations the micropillars are present in a vessel component of a microfluidic device, such as a chip, and are spaced such that they have a center-to-center distance of about 50 μm. In embodiments interpillar distance is about 30 μm. A representative and non-limiting image of a scanning electron micrograph of a cross section of a chip comprising micropillars is presented in FIG. 17. In FIG. 18, scale bars of 20 μm width (width of second pillar from left), 30 μm width (width between second and third pillar from left) and 50 μm width (distance between center of said micropillars) are shown. The scale bar in the bottom right represents 100 um in length. The pillars are staggered, so those in light gray are the nearest to the front, while those in dark gray are recessed. Micropillar height is as depicted as 189 and 199 μm. Variations in any of these dimensions are encompassed by the disclosure. For example, the total sample volume capacity on the chip can be modified by increasing the chip area, e.g., its footprint, and/or by modifying the depth of cavities between the micropillars according to accommodate any particular sample volume. In embodiments, non-limiting examples of depth are from 100 μm-500 μm. In embodiments the depth can be from 300-350 μm. In embodiments the cavity depth can be up to 350 μm.

After micropillars are formed, regardless of the particular technique(s) used in their formation, the micropillars are coated with a suitable material, such as the silicon oxides or metal oxides as described herein. In certain embodiments the micropillars are coated with, for example, Al₂O₃, HfO₂. Si₃N₄, or SiO₂, using any suitable approach. In one approach chemical vapor deposition (CVD) is used. CVD techniques are known in the art and given the benefit of the present disclosure can be adapted for use in embodiments of this disclosure to, for example, coat micropillars with Al₂O₃, HfO₂, Si₃N₄. In certain approaches the micropillars can be coated with SiO₂ using approaches that are distinct from CVD, such as by thermal oxidation of silicon. Those skilled in the art will readily be able to adapt well known parameters of oxide growth kinetics during thermal oxidation of silicon to achieve suitable SiO₂ micropillar coating. In embodiments, micropillars of this disclosure comprise an outer layer of Al₂O₃, HfO₂, Si₃N₄, or SiO₂ that has a thickness of 10-500 nm, inclusive, and including all numbers and ranges of numbers there between.

As discussed above, the micropillars may be present in a vessel. Any suitable vessel can be employed such that samples comprising nucleic acids as described herein can be isolated, and/or purified, and/or analyzed. Generally the vessel has any shape that permits fluid sample flow, including but not necessarily limited to a straight vessel, a vessel having bends comprising corners, or curved bends. In embodiments the vessel has a serpentine shape, and thus has one or more bends or meanders. In non-limiting embodiments a serpentine vessel has from 4-12 bends. The vessel has a total fluid volume capacity that can vary depending on the particular implementation and by changing the area of its footprint and/or its depth. In general a suitable vessel has a fluid capacity volume of not less than 1 μL. In non-limiting examples the vessel fits into an area of from 4.0 mm×6.0 mm, inclusive and including all numbers and ranges of numbers there between. In an embodiment the disclosure includes a vessel comprising a pillar chamber that is approximately 4.5 mm×5.0 mm. As one non-limiting illustration, a schematic of a vessel design is shown in FIG. 18, with an exploded view of the vessel surface in the lower right corner, which is provided in connection with the electron micrograph depicted in FIG. 17.

In certain examples used herein, micropillars formed as described above are scraped from the wafer in which they are formed. The scraped micropillars are then used in solution to perform a nucleic acid assay that is designed to mimic an internal vessel environment in terms of volume, buffer components, amplification and detection reagents, time, temperature, micropillar density, surface area, pH, etc.

In various embodiments the disclosure includes any surface(s) described herein, wherein the surfaces are in non-covalent association with nucleic acids. In embodiments the disclosure comprises a plurality of micropillars coated with a composition comprising or consisting essentially of Al₂O₃, HfO₂, Si₃N₄, or SiO₂ as described herein, wherein the polynucleotides are in a non-covalent physical association with the surface of the micropillars, i.e., the polynucleotides are in a complex with the micropillars. Those skilled in the art will recognize that the physical associations/complexes as described herein may be transient and are subject to thermodynamic, fluid dynamic, biochemical factors, buffering conditions, equilibriums, etc., that may be present during the performance of any nucleic acid isolation, detection, quantitation or quantification process of this disclosure.

The solutions, also referred to herein as buffers, used in the present disclosure include one or more of each of: a lysing buffer, a binding buffer, a wash buffer, and an elution buffer. In certain embodiments, the binding/lysing solution has an acidic buffered pH (e.g., 0, 1, 2, 3, 4, 5, 6, or 7), includes one or more salts, including kosmotropic salts and NaCl, and optionally a nuclease inhibitor and/or a proteinase. In certain embodiments, this solution has a pH of between about 1 and 5. For example, the solution may have a pH of about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, and 5.0. In certain embodiments, the solution has a pH from about 2.0 to 4.0. In one embodiment, the solution has a pH from about 2.5 to 3.9. In certain embodiments, the salt constituents include NaCl from about 0.5M to 2.0M (e.g., 0.5M, 0.6M, 0.7M, 0.8M, 0.9M, 1.0M, 1.1M, 1.2M, 1.3M, 1.4M, 1.5M, 1.6M, 1.7M, 1.8M, 1.9M, and 2.0M), and kosmotropic salts from about 0.01M to 5M. For example, the kosmotropic salts may have a concentration of about 0.01M, 0.02M, 0.03M, 0.04M, 0.05M, 0.06M, 0.07M, 0.08M, 0.09M, 0.1M, 0.11M, 0.12M, 0.13M, 0.14M, 0.15M, 0.16M, 0.17M, 0.18M, 0.19M, 0.2M, 0.21M, 0.22M, 0.23M, 0.24M, 0.25M, 0.26M, 0.27M, 0.28M, 0.29M, 0.3M, 0.31M, 0.32M, 0.33M, 0.34M, 0.35M, 0.36M, 0.37M, 0.38M, 0.39M, 0.4M, 0.41M, 0.42M, 0.43M, 0.44M, 0.45M, 0.46M, 0.47M, 0.48M, 0.49M, 0.5M, 0.6M, 0.7M, 0.8M, 0.9M, 1.0M, 1.1M, 1.2M, 1.3M, 1.4M, 1.5M, 1.6M, 1.7M, 1.8M, 1.9M, 2.0M, 2.1M, 2.2M, 2.3M, 2.4M, 2.5M, 2.6M, 2.7M, 2.8M, 2.9M, 3.0M, 3.1M, 3.2M, 3.3M, 3.4M, 3.5M, 3.6M, 3.7M, 3.8M, 3.9M, 4.0M, 4.1M, 4.2M, 4.3M, 4.4M, 4.5M, 4.6M, 4.7M, 4.8M, 4.9M, and 5.0M. In certain embodiments, the kosmotropic salts have a concentration of about 0.1M to 3M. In one embodiment, the kosmotropic salts have a concentration of about 0.1 to 1.0 M. In one embodiment, the kosmotropic salts have a concentration of about 0.1M to 0.35M. The nuclease inhibitor constituent prevents or reduces the contamination of the purified nucleic acids by ribonucleases that could degrade the purified nucleic acid, and include, for example PROTECTOR RNase Inhibitor (Roche), SUPERaseIn™ (ThermoFisher Scientific), RNaseOUT™ (ThermoFisher Scientific), RNase Inhibitor (ThermoFisher Scientific) and RNasin™ (Promega). In certain embodiments, the concentrations of inhibitor are from about 1-2 U/μL. For example, the concentration of the inhibitor may be about 1.0, 1.1, 1.2, 1.3, 1.4, 1.5, 1.6, 1.7, 1.8, 1.9, or 2.0 U/μL. The proteinase constituent denatures and degrades protein contaminants and proteins released through lysis that would interfere with nucleic acid amplification, and include, for example, Proteinase K (Geriaid), also known as Peptidase K or Endopeptidase. In certain embodiments, the concentrations of the proteinase are from about 0.001-100 U/μl. In one embodiment, the concentration of the proteinase is from about 0.01-10 U/μl. Kosmotropic salts provide cations or anions that contribute to the ordered stability of a polar solvent (e.g., water). In certain embodiments, the kosmotropic salts are included in some solutions or buffered solutions. The kosmotropic salts may be, for example, sulfates, acetates, carbonates, and phosphates, such as (NH₄)₂SO₄ (ammonium sulfate), CH₃COONa (sodium acetate), H₂CO₃ (carbonic acid and its basic species), and K₂HPO₄ (potassium phosphate and its acidic/basic species). In embodiments the methods disclosed herein minimize or entirely avoid the use of the organic solvents and other chaotropic agents that are commonly used in nucleic acid purification. Without intending to be bound by any particular theory it is considered that these components are not suitable for use outside of clinical or laboratory settings, would increase cost of manufacture, and would otherwise require special shipping/handling of a finished point of care device.

In some embodiments, the one or more wash solutions include at least a first wash buffer and a second wash buffer. Wash buffers function to remove lysing debris and other contaminants from the nucleic acids after the nucleic acids have adhered to the surface. In certain embodiments, the first wash buffer is mildly acidic and contains kosmotropic salts, a reducing agent and may include, for example, an RNase inhibitor. In one embodiment, the first wash buffer has a pH below 7. For example, the first wash buffer may have a pH of about 0.0, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, or 6.9. In certain embodiments, the first wash buffer has a pH below about 5. In one embodiment, the first wash buffer has a pH below about 4. Kosmotropic salts may be selected as described above, and may be present in a concentration of from 0, 1M to 5 M. For example, the kosmotropic salt may have a concentration of from about 0.1M, 0.11 M, 0.12M, 0.13M, 0.14M, 0.15M, 0.16M, 0.17M, 0.18M, 0.19M, 0.2M, 0.21M, 0.22M, 0.23M, 0.24M, 0.25M, 0.26M, 0.27M, 0.28M, 0.29M, 0.3M, 0.31M, 0.32M, 0.33M, 0.34M, 0.35M, 0.36M, 0.37M, 0.38M, 0.39M, 0.4M, 0.41M, 0.42M, 0.43M, 0.44M, 0.45M, 0.46M, 0.47M, 0.48M, 0.49M, 0.5M, 0.6M, 0.7M, 0.8M, 0.9M, 1.0 M, 1.M, 1.2M, 1.3M, 1.4M, 1.5M, 1.6M, 1.7M, 1.8M, 1.9M, 2.0M, 2.1M, 2.2M, 2.3M, 2.4M, 2.5M, 2.6M, 2.7M, 2.8M, 2.9M, 3.0M, 3.1M, 3.2M, 3.3M, 3.4M, 3.5M, 3.6M, 3.7M, 3.8M, 3.9M, 4.0M, 4.1M, 4.2M, 4.3M, 4.4M, 4.5M, 4.6M, 4.7M, 4.8M, 4.9M, to about 5.0M. In certain embodiments, the kosmotropic salt has a concentration of from about 0.2 to 4.0 M. In some embodiments, the kosmotropic salt has a concentration of from about 0.5 to 2.5M. Reducing agents of the present disclosure include, but are not limited to DTT (dithiothreitol) and TCEP (tris(2-carboxyethyl)phosphine). The reducing agent may be present at a concentration from about 0.1 mM to about 20 mM. For example, the reducing may be present at a concentration from about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, to about 20 mM. In some embodiments, the reducing is present at a concentration from about 1 to 10 mM. In some embodiments, the reducing is present at a concentration from about 1 to 10 mM, with an RNase inhibitor as selected and described above.

Additional wash buffers may differ in pH and other properties, for example, possess lower or higher ionic strength, lower or high pH, or lower or higher concentrations of kosmotropic salts or RNase inhibitor, with adjustments in composition and concentration of solutes made to improve the purification of the adhered nucleic acids without reducing their adherence to the surfaces. A second wash buffer with lower ionic strength may be used to remove additional contaminants or residual salts remaining from the first wash buffer. In certain embodiments, the second wash buffer is mildly acidic, contains kosmotropic salts, and optionally, lower amounts of reducing agent and lower amounts of RNase inhibitor, if any. In some embodiments, the second wash buffer has a pH below 7. In one embodiment, the second wash buffer has a pH below pH 5. In some embodiments, the second wash buffer has a pH of about 3.5 to 4.5. Kosmotropic salts may be selected as described above, and may be present in a concentration of from 0.1 to 50 mM. For example, the kosmotropic salt may have a concentration of about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mM. In some embodiments, the kosmotropic salt has a concentration of about 1 to 40 mM. In some embodiments, the kosmotropic salt has a concentration of about 10 to 25 mM. For example, a second wash buffer that has a pH of about 4.0 buffered with 10 to 25 mM ok a kosmotropic salt (e.g., NaOAc) may be employed.

The elution buffer (which may also function as the amplification buffer) dislodges the nucleic acids from the surface. For methods where amplification is performed while the nucleic acids are adhered, the elution buffer is administered following amplification. Where amplification is performed in solution, the elution buffer is used to dislodge the nucleic acids, with the amplification reagents added at the same time or thereafter. In certain embodiments, amplification and purification are performed in the same microfluidic space or reactor. In certain embodiments, the elution buffer has a low ionic strength and a neutral to slightly basic pH. For example, an elution buffer may have a pH from 6 to 10. For example, the elution buffer has a pH of 6, 7, 8, 9, or 10. In some embodiments, the elution buffer as a pH of about 8.5. In one embodiment, the elution buffer as a pH of about 8.5, contains about 10 to 25 mM Tris (Tris(hydroxymethyl)aminomethane), and from about 0% to 35% DMSO (dimethylsulfoxide). Without being bound by any particular theory, 0% to 35% refers to v/v. In some embodiments, the elution buffer contains about 5 to 25% DMSO, as a stabilizer. Thus, the elution buffer/amplification buffer in certain embodiments may be free of DMSO or may contain only trace amounts of DMSO. In some embodiments, the disclosure comprises lysing viral and/or cellular components and binding nucleic acids to a surface in a single step, and the same solution is thus used in the binding step and lysing steps, as this increases the speed of the overall purification, amplification and detection of the nucleic acids.

In some embodiments, the disclosure facilitates detection of threshold amounts of nucleic acids in the sample that is tested. As a non-limiting and hypothetical example, if a sample comprises 100 copies of a viral genome, in embodiments the disclosure is suitable for detecting at least 1 of the copies of the genome, and can comprise detecting from 4-100 of the copies, inclusive, and including all numbers and ranges of numbers there between. In some embodiments, the disclosure comprises generating a positive result with as few as four copies of a nucleic acid, such as an HCV genome. In some embodiments, from 1×10⁷ to 4×10⁷ copies are detected.

While non-limiting demonstrations of this disclosure are provided using plasma comprising known RNA inputs, it is expected that any biological sample, or other sample that comprises, or is or could be suspected to comprise, or is known to comprise nucleic acids, can be used in embodiments. In some implementation the sample comprises environmental samples, such as samples of water, food substances, or samples taken from an inanimate object, or an inanimate surface, including but not limited to devices or other flat or three-dimensional objects, devices, etc. In embodiments the sample is a biological sample. The biological or other sample may be used directly or it may be subjected to a processing step before being applied to a device of this disclosure. In embodiments the biological sample comprises a liquid biological sample, including but not limited to blood, plasma, urine, cerebrospinal fluid, lymph, saliva, sweat, semen, and lacrimal secretions. The biological sample may be a processed solid biological sample, such as a biopsy that has been subject to a mechanical disaggregation and/or may be subjected to one or more solutions. The biological sample may be obtained from a human or a non-human mammal or an avian animal using any suitable technique. Thus, in certain aspects the disclosure is pertinent to diagnostic applications in the field of veterinary medicine, in addition to human medical applications.

The polynucleotides isolated/amplified/detected/quantitated/quantified using approaches of this disclosure are not particularly limited. In general the polynucleotides will be of adequate length such that they are subject to amplification, including but not necessarily limited to amplification by methods that involve a polymerase chain reaction (PCR), including but not limited to Real-Time PCR (RT-PCR), i.e., quantitative RT-PCR (qPCR), as described further herein. The polynucleotides may be single or double stranded, or partially single or double stranded, and may be RNA or DNA. In some embodiments, the polynucleotides are RNA molecules, and their amplification can include a reverse-transcriptase for cDNA generation and further amplification and/or quantification. The type and/or origin of the nucleic acid that is determined using embodiments of this disclosure is not particularly limited and can come from, for example, any microorganism, which include but are not necessarily limited to pathogenic microorganisms. The microorganisms may be prokaryotic or eukaryotic. “Microorganisms” for the purposes of this disclosure also comprise viruses. In embodiments the microorganisms are selected from fungi, bacteria, archaca, viruses, and protozoans, including parasitic protozoans. In embodiments the disclosure relates to identifying nucleic acids from pathogenic bacteria. It is considered the disclosure can be used with any genus, species, or strain of bacteria. In certain examples the disclosure is used to detect nucleic acids from intracellular parasites.

With respect to viruses, while it is expected that any virus can be analyzed, in certain embodiments the virus is characterized by a single stranded RNA genome. The genome may be a (+) strand or a (−) strand. The viruses may be enveloped or non-enveloped. In embodiments the viral RNA is a viral genome from the viral family Filoviridae, or Paramyxoviridae, or Rhabdoviridae, or Bunyaviridae, or Arenaviridae, or Orthomxroviridae (including all types of influenza viruses). In embodiments the viral RNA is a viral genome or fragment thereof from the viral family Picornaviridae, Astroviridae, Caliciviridae, Hepeviridae, Flrvivindae, Togavindae, Arteriviridae, and Coronaviridae. In specific embodiments the viral RNA is from a human immunodeficiency virus (HIV), a hepatitis A virus, a hepatitis B virus, a hepatitis C virus, a cytomegalovirus, a human lymphotropic virus, an Epstein-Barr virus, a parvovirus, a paramyxovirus, or a herpes simplex virus. In other embodiments the RNA is an mRNA, or is a non-coding RNA. In embodiments the RNA comprises a snoRNA, an miRNA, or a mitochondrial RNA. In certain embodiments the RNA may be initially present in the biological sample as a component of a membranous vesicle, including but not limited to an exosome. In embodiments the RNA may be a circulating RNA of any type that is indicative of a disorder, including but not necessarily limited to cancer. Non-limiting embodiments of this disclosure are illustrated using HCV, HIV, Zika, HPV-16, and HPV-18.

In certain non-limiting embodiments a biological sample comprising, or suspected of comprising, a polynucleotide is subjected to a composition or process that is intended to disrupt the cell, virus or other substance in which the polynucleotides to be analyzed may be present. In certain embodiments, disruption comprises lysis of a cell or disrupting cell membranes, and/or disrupting a viral particle such that nucleic acids within the cell or the viral particle become amenable to binding to a surface of this disclosure. In non-limiting embodiments the sample is subjected to a chemical treatment, a thermal treatment, or a mechanical treatment (including but not limited to sonication) or a combination thereof, such that nucleic acids in the samples if present become, or are prepared to become, accessible to surfaces of this disclosure. In certain embodiments, lysis is performed using a chemical treatment that may include, for example, any of a variety of components which include but are not limited to detergents. Suitable detergents are known in the art and include for example SDS, which can be used at any suitable concentration, such as 1%, and NP-40, which can be used at for example, 0.5%. In certain approaches the sample can be subjected to a processing step in a device component, such as a cartridge, wherein the sample is exposed to any one or combination of the aforementioned compositions and/or conditions. The sample may also be subjected to, for example, a mechanical pressure that causes a fluid component of the sample to pass through a separation material such as a membrane having any suitable degree of porosity. The mechanical pressure may be adequate to pass some or all of the sample volume into and/or partially or fully through a microfluidic vessel described herein. In embodiments, the sample travels through the device without mechanical pressure and instead migrates via capillary action. In embodiments a wicking material can be included. In embodiments the sample can be subjected to heat that is provided by any suitable source or apparatus, for example, by an on-board exothermic chemical reaction component. The same approach can be adapted for heating that occurs, for example, during amplification reactions, and the process may further employ endothermic chemical reaction components for cooling purposes—thus, in certain embodiments a device of this disclosure can operate independent of batteries or other sources of electric power, further providing advantages for point of care applications in a wide variety of settings, including but not necessarily limited to the scene of a medical emergency, including but not limited to a battle-field environment.

In certain embodiments a result obtained from using a method and/or device and/or system of this disclosure can be compared to any suitable reference, examples of which include but are not limited control sample(s), a standardized curve(s), and/or experimentally designed controls such as a known input polynucleotide value used to normalize experimental data for qualitative or quantitative determination of the amount of polynucleotide, or a cutoff value, such controls being useful if desired to normalize for mass, molarity, concentration and the like. A reference value may also be depicted as an area on a graph. In embodiments the disclosure provides for an internal control that can be used to normalize a result, such as a signal that indicates an amount of nucleic acids. In embodiments the disclosure provides for use of calibrators, i.e., known inputs which can be used to test, establish, confirm, etc. the accuracy of any particular signal. In certain embodiments one or more calibrator and/or internal control samples can be stored on-chip, or they can be stored in a separate device component, including but not necessarily limited to a permanently fixed and/or detachable cartridge component. In certain aspects the disclosure includes calibrating each chip, or calibrating only selected chips from a group (i.e., a lot) of chips. In certain non-limiting examples, the disclosure includes a positive control and/or a calibrator in a distinct channel or other segment of a chip. In one implementation a lysis/binding buffer can pass over a segment where a known concentration of control RNA, which may comprise so-called armored RNA (comprising for example a complex of bacteriophage protein and RNA) is solubilized in the lysis/binding buffer and delivered to an extraction/amplification chamber. The armored RNA can be lysed, bound to pillars as described herein, washed and amplified/detected in the same way as the test sample. In embodiments a Ct value of the control will be used for comparison to a pre-determined standard curve that is associated with a specific lot of chips, and thus the standard curve can be adjusted based on the Ct value of the control, such as if it falls within a pre-defined range. Additionally or alternatively, embodiments of the disclosure can include an internal control, such as any suitable in vitro transcribed RNA if the sample is being tested for RNA, that can be used to assess assay parameters, performance, etc. In a non-limiting embodiment the internal control RNA comprises in vitro transcribed moss gene RNA. Such an internal control RNA will be subjected to analysis in both test and positive control channels and thus will be extracted, amplified and detected at the same time as test RNA, which can be performed in a multiplexed format. The internal control probe may be conjugated with a fluorophore or other detectable label that is spectrally distinct from the test probe fluorophore. Accordingly, increased fluorescence from both probes can be monitored concurrently and configured such that the internal control must fall within a pre-defined range for the test result to be considered valid.

In certain embodiments a result based on a determination of the presence, absence, or amount of a polynucleotide using an approach of this disclosure is obtained and is fixed in a tangible medium of expression, such as a digital file, and/or is saved on a portable memory device, or on a hard drive, or is communicated to a web-based or cloud-based storage system. The determination can be communicated to a health care provider for diagnosing or aiding in a diagnosis, such as of a bacterial or viral infection, or for monitoring or modifying a therapeutic or prophylactic approach for any disease, disorder or condition that is associated with the presence and/or amount of the polynucleotide in the sample.

In certain examples the disclosure comprises an article of manufacture, which in embodiments can also be considered kits. The article of manufacture comprises at least one component for use in the nucleic acid analysis approaches described herein and packaging. The packaging can contain a device and/or a chip comprising microfluidic vessels described herein. In various embodiments, the article of manufacture includes printed material. The printed material can be part of the packaging, or it can be provided on a label, or as paper insert or other written material included with the packaging. The printed material provides information on the contents of the package, and instructs user how to use the package contents for nucleic acid analysis. In embodiments the article of manufacture can comprise one or more suitable sealed, sterile containers that contain for example, buffers described herein or stock solutions, primers that are directed to known polynuclcotide sequences for any particular organism, primers for use in RT-PCR reactions, labeled probes for use in such reactions, enzymes, such as reverse transcriptase and a suitable DNA polymerase, RNAse inhibitors, nucleotides, etc. In one approach the package comprises a cartridge comprising one or more buffers used in nucleic acid extraction and/or for annealing nucleic acids to a surface in a device component.

In one example of the present disclosure, buffer, blood plasma or other biological specimen containing nucleic acids is added to the binding buffer, the mixture is heated to about 50 to 70° C. and incubated with a metal oxide- or silicon oxide-coated surface as described above to allow for nucleic acid binding. Taking advantage of the micro/nano fluidic scale, incubation times are, in certain embodiments, no more than 60 minutes. In some embodiments, incubation times are no more than 45 minutes. In some embodiments, incubation times are no more than 30 minutes. In some embodiments, incubation times are no more than 20 minutes. In some embodiments, incubation times are no more than 15 minutes. In some embodiments, incubation times are between about 1 and 15 minutes. For example, the incubation times are between about 1, 2, 3, 4, 5, 6, 7, 8, 9, 10, 11, 12, 13, 14, and 15 minutes. Following this incubation, the surface is washed with a first wash buffer in order to remove contaminants. Next, the surface is washed with a second wash buffer to remove residual wash salts. Finally, the nucleic acid may be eluted from the surface using an elution buffer (and incubation at 55° C.) or further steps (i.e. amplification) may be performed with the nucleic acids still adhered to the surface.

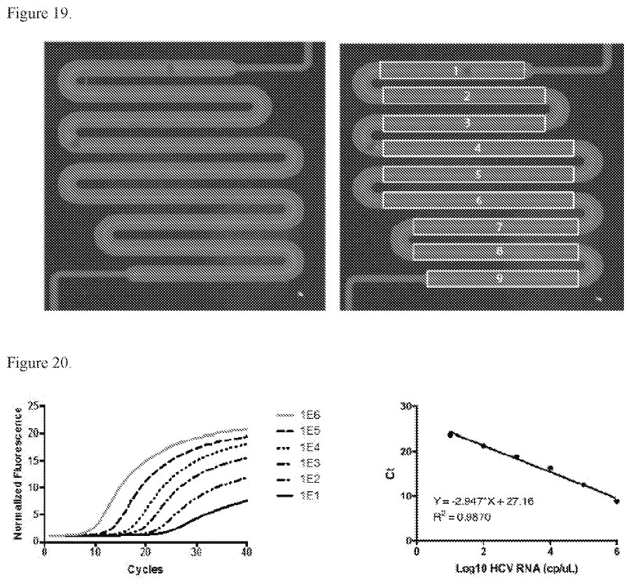

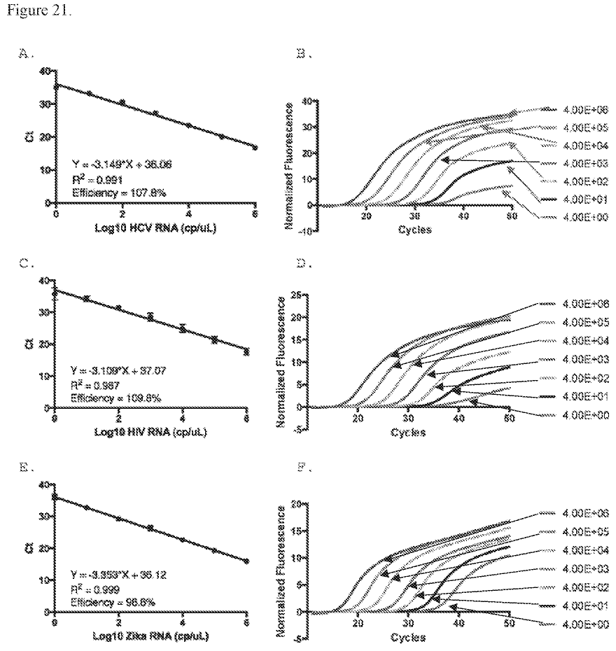

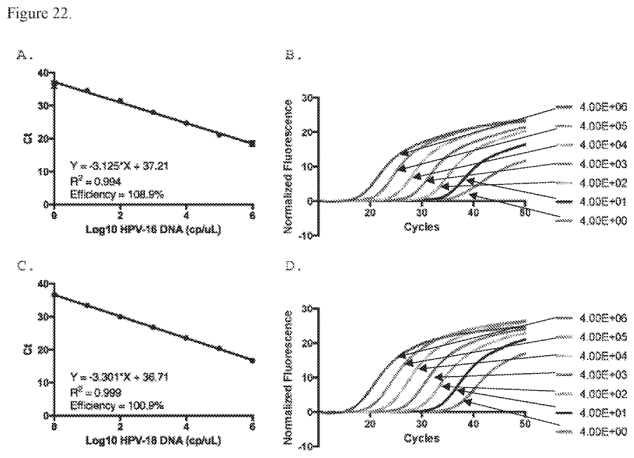

In certain embodiments the fluorescence emission can be measured in one or more segments of the reaction chamber, as illustrated in FIG. 19. The device may be provided with or without an optically transparent window, which may be comprised of any suitable material such as silicon (i.e., silicon on silicon). In embodiments, the disclosure provides for readout of a signal using a reaction chamber that is optically accessible (e.g. quartz on silicon or other transparent material), such as with an imager located proximal to the reaction chamber. In embodiments the fluorescence is detected within the vessel, thus the vessel can function akin to a fiber optic conduit. Thus, the disclosure encompasses in alternative embodiments use of so-called free-space optics to detect a signal via any suitable signal detection device that is placed in proximity to the location where the signal is generated, or the use of an optical waveguide to transmit the signal to any suitable measuring device such that optical accessibility to the reaction chamber is not necessarily required to detect the signal. In certain aspects the disclosure encompasses stimulating a fluorophore with an excitation light such that light having an emission wavelength is generated. In embodiments, the excitation light and emission light travel through an optical waveguide to a detection device such that the emission is signal is detected. Any suitable waveguide material can be used. In embodiments the optical waveguide is formed of silicon nitride. In embodiments an optical waveguide can be integrated into a chip of this disclosure. In embodiments the optical waveguide comprises a waveguide formed of silicon oxide (SiO₂), titanium oxide (TiO₂), glass, or any of a variety of polymers which include but are not necessarily limited to polymethylmethacrylate (PMMA).

In embodiments, one or more segments of the vessel can be connected to or in communication with a digital processor and/or a computer running software to interpret the position, amount, intensity, etc. of the fluorescent signal. A processor may also be included as a component of a device comprising the chip, wherein the processor runs software or implements an algorithm to interpret fluorescence or another optically detectable signal, and generate a machine and/or user readable output. In an embodiment, a chip component can be integrated or otherwise inserted into an adapter that comprises a detection device, such as a camera, which may also comprise a processor for fluorescence detection. In embodiments, a computer readable storage media can be component of a device of this disclosure, and can be used during or subsequent to performing any assay or one or more steps of any assay described herein. In embodiments the computer storage medium is a non-transitory medium, and thus can exclude signals, carrier waves, and other transitory signals.

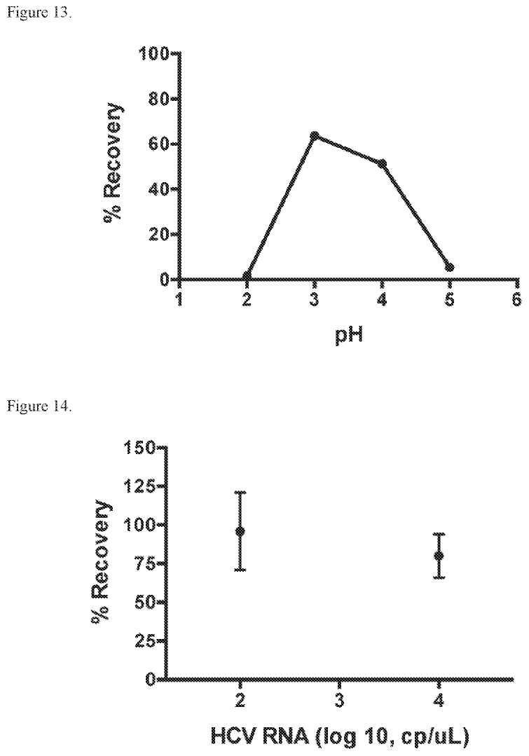

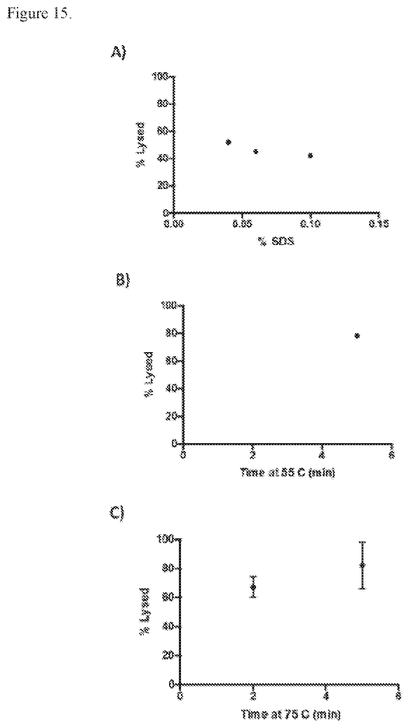

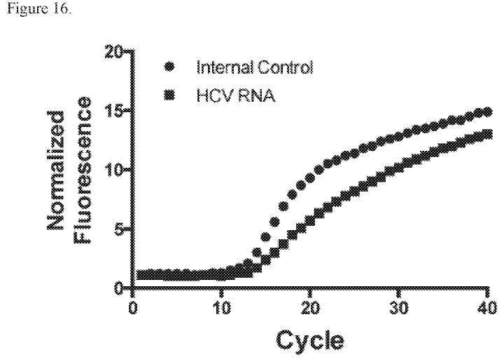

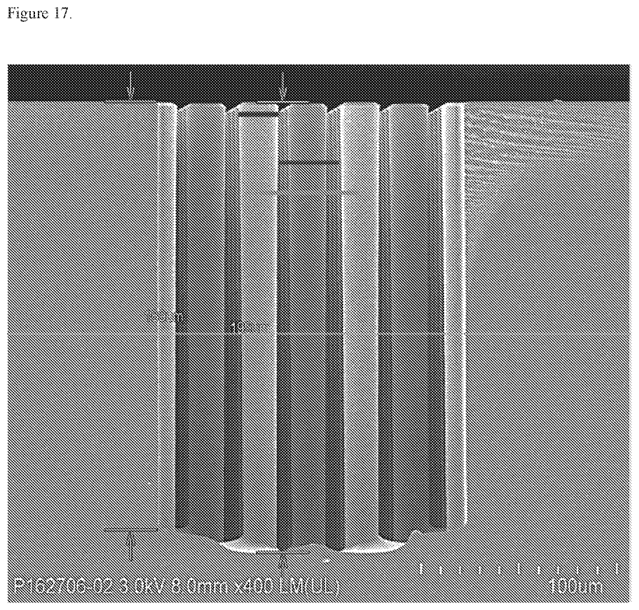

Examples 1 and 2 describe RT-qPCR assays designed as a basis for advancing embodiments of the disclosure to on-chip applications. Examples 3 and 4 were performed using blanket silicon wafers coated with hafnium oxide or aluminum oxide, the blankets comprising a diced rectangular piece of silicon wafer containing wells formed by an attached gasket. The blanket is a model for the surface of a microreactor of this disclosure. RNA is bound to the blanket, washed, eluted and quantified in a benchtop RT-qPCR. Examples 5, 6 and 7 were performed using silicon nanopillars scraped from a microfluidic reactor, where the nucleic acids adhered to pillar structures formed as described above. The pillars are representative of pillar surface coatings in a commercial embodiment of a reactor and were less dependent upon diffusion and higher recoveries. Notably, results in Examples 6 and 7 were obtained using RNA bound to silicon oxide-coated pillars; the RNA was not eluted off the pillars before quantification, thus supporting on-chip implementations using nanopillar coated surfaces. Example 8 demonstrates extraction of RNA by capillary flow (FIG. 14), chemical lysis of HCV particles and quantification of the percent of particle lysis in different SDS concentrations and temperatures (FIG. 15) and use of an internal control RNA to normalize results obtained in RT-qPCR reactions. The disclosure further includes data showing fluorescence normalization using an internal control RNA and HCV RNA (FIG. 16) and also demonstrates on-chip amplification of HCV RNA (FIG. 20).

The following Examples are meant to illustrate but not limit embodiments of this disclosure. For Examples 1 and 2, an HCV (Hepatitis C Virus) RNA detection assay was performed with a sensitivity of 4 copies (cp) per reactions. Multiple nucleic acid amplification assays were tested across a standard concentration range of 4×10⁰-4×10⁶ cp/reaction. These standard concentrations were selected based on the range of HCV RNA plasma concentrations measured in 98% of HCV-infected patients and performance specifications of upstream steps, plasma separation and RNA extraction. [See, for example, Ticehurst, et al. J Clin Microbiol. 2007 August; 45(8): 2426-2433.] These specifications included a maximum blood sample of 20 μL, 50% recovery of plasma from blood sample (blood contains ˜50% plasma, on average), and 40% recovery of RNA during the extraction process. Table 1 illustrates, using representative high and low-end copy number values along the spectrum of observed patient HCV RNA concentrations, the number of HCV RNA copies in each step along the process taking into account these specifications.

TABLE 1
Copies of RNA in intermediate steps in NA process.

| Example HCV RNA sample | High | Low |
|---|---|---|
| Patient RNA plasma concentration (cp/μL) | 7.0 × 10⁷ | 2.0 × 10³ |
| RNA in 10 μL plasma from 20 μL blood sample | 7.0 × 10⁵ | 2.0 × 10¹ |
| (cp) | | |
| RNA in recovered 5 μL plasma (50% recovery, | 3.5 × 10⁵ | 1.0 × 10¹ |
| cp) | | |
| Extracted RNA to be used in RT-qPCR assay (cp) | 1.7 × 10⁵ | 4.0 × 10⁰ |

Thus, these assays had sufficient dynamic range (4×10⁰-4×10⁶ cp/reaction) to detect 98% of all HCV RNA-positive specimens. Testing was performed using an in vitro transcribed, HCV 5′ UTR RNA standard, described below, diluted to 1×10⁰-1×10⁶ cp/μL. Ten replicates of each sample were analyzed during performance testing.

Two RT-qPCR assays were screened for pretesting prior to use on microfluidic purification, amplification and detection. Assay (A) Superscript III-Tfi (Example 1) and Assay (B) Superscript III-Amplitaq360 (Example 2). Both assays utilize two enzymes: 1) MMLV-based reverse transcriptase (Superscript 111) that has been genetically engineered for a longer half-life at higher reaction temperature and decreased RNascH activity and 2) DNA polymerase cloned from either Thermus filiformis (Tfi) or Thermus aquaticus (AmpTaq360) that has been genetically engineered for enhanced processivity and stability. Both the reverse transcription (RT) and qPCR reactions are performed in the same well on a 96-well plate instead of performing the RT reaction in one well and then transferring a portion of the reaction to a qPCR well in order to model an RT-qPCR assay performed in a single reaction chamber on silicon microchip. In this “one-tube” reaction, the temperature is maintained at an optimal temperature for generation of cDNA (RNA to single-stranded DNA) by the RT enzyme. The temperature is then increased to inactivate the RT enzyme and activate the DNA polymerase. The cDNA is then amplified using normal PCR temperature cycling—high temperature to melt DNA duplexes and lower temperature to elongate DNA from annealed oligonucleotide primers. The amplified DNA is quantified through the use of a fluorescently labelled oligonucleotide probe. The probe comprises a sequence that is complementary to the target sequence, a fluorophore conjugated to the 5′ end, and a quencher molecule conjugated to the 3′ end. During qPCR amplification, the inherent 5′-3′ exonuclease activity of the DNA polymerase results in digestion of the probe in a 5′-3′ direction, release of the fluorophore, and alleviation of quencher-mediated suppression of the 5′ fluorophore fluorescence. The fluorescence is measured at the end of each qPCR cycle and compared to values obtained from standards in order to quantify the concentration of RNA in the sample. For both assays, an in vitro transcribed RNA standard was used to test the performance of each assay across a standard range of 4×10⁰-4×10⁶ cp/reaction. The RNA standard was supplied from Amsbio and produced from a plasmid template that contained the complementary full-length sequence of the HCV 5′ UTR region obtained from a standard lab isolate. The HCV 5′ UTR is a highly conserved region of the HCV genome so use of this sequence from this isolate should be representative of the natural diversity of HCV sequences. The RNA is produced using standard in vitro transcription techniques and template DNA is digested using DNase. The resulting product contains <0.01% template DNA contamination. The DNA contamination level is acceptable because this amount of DNA will not significantly contribute to signal generation and the standard is DNA free at concentrations <1×10⁴ cp/μL.

### Example 1

This Example provides a description of analysis using Superscript III-Tfi RT-qPCR.

Reverse transcriptase: MMLV-based Superscript III (SSIII) supplied by Invitrogen; DNA polymerase: Thermus filiformis polymerase (Tfi) supplied by Invitrogen. Final reaction reagent concentrations: 40 mM Hepes-KOH (pH 8.1), 15 mM KCl, 15 mM (NH₄)₂SO₄, 2% glycerol, 200 nM dNTP, 200 nM forward primer (5′-CCCCTGTGAGGAACTACTGT-3′), 400 nM reverse primer (5′-GACCACTATGGCTCTCCCG-3′), 200 nM Atto633-conjugated probe (5′-Atto633-AGCCATGGCGTTAGTATGAGTGTCG-IAbRQSp-3′), 3.0 mM MgCl₂, 0.2 mg/mL. BSA, 3 mM DTT, 50 U SSIII, and 1.7 U Tfi polymerase.

A 1.67× concentrated master mix was prepared and 6 μL of this master mix was added to 4 μL of RNA. For this experiment, standards were diluted in 10 mM Tris pH 7.5 containing ng/μL carrier RNA to final concentrations ranging from 1×10⁰-1×10⁶ copies/μL. Temperature cycling parameters on LIGHTCYCLER® 480 (Roche Life Sciences) instrument were 55° C. for 15 min, 95° C. for 3 min, 50 cycles of 1) 95° C. for 5 sec, 2) 60° C. for 30 sec. Fluorescence was only monitored during cycles 11-50.

Data analysis—Raw fluorescence values from the LIGHTCYCLER® 480 (Roche Life Sciences) instrument were used to fit amplification curves using the R qpcR package (freeware statistical analysis program). Ct values for each replicate were assigned by determining the 2^{nd} derivative maximum value for a 5-parameter sigmoidal fit curve. Average Ct values for each standard were plotted against the Log 10 concentration for each standard and the data were fit with a linear regression line. The slope of the line was used to determine reaction efficiency (efficiency=10^{(-1/slope)}−1) and the back-calculated concentrations of each standard replicate were used to estimate sample reproducibility (standard deviation) of each standard dilution.

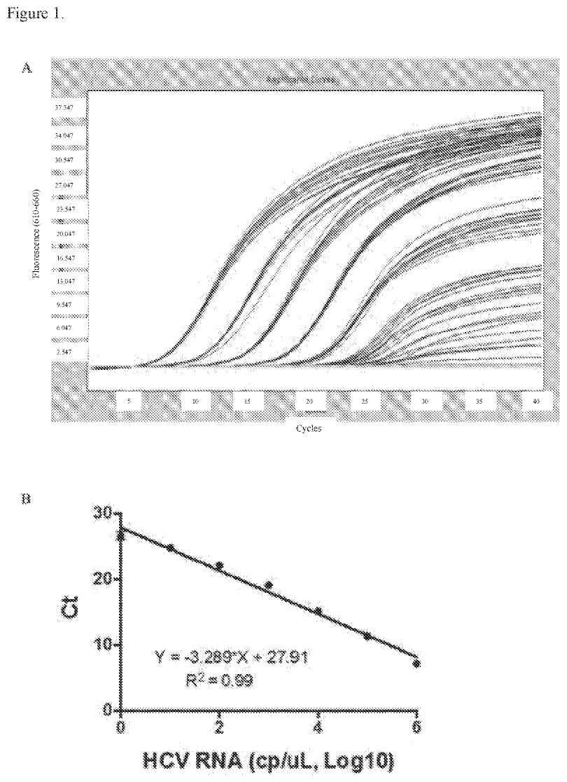

For validation of this assay, 10 replicates of each standard (1×10⁰-1×10⁶ cp/μL) were analyzed on the LIGHTCYCLER® 480 (Roche Life Sciences) instrument using the method described above, amplification curves analyzed using the R qpcR package, standard curves generated, and assay performance was evaluated, as described above. FIG. 1 illustrates the amplification curves and the standard curves for the Superscript III-Tfi RT-qPCR performance test. The equation of the linear regression line is included in the standard curve graph. Ten replicates of each standard dilution were analyzed using the SSIII-Tfi RT-qPCR assay and the amplification curves for the test are depicted in FIG. 1. The mean Ct values for each standard were plotted against standard RNA concentration and the data were fit with a linear regression line with the following equation: Y=−3.289×+27.91 (FIG. 1 B). This slope corresponds to a reaction efficiency of 101.4%. All 10 replicates of the 4×10¹-4×10⁶ cp/reaction standards (100% detection) and 8 replicates of the 4×10⁰ cp/reaction standard (80% detection) were detected (Table 2). While the assay reproducibility decreased (represented by an increasing variability) with decreasing concentration of HCV RNA standard, the average reproducibility across all standards was 0.09 Log 10 (Table 2).

TABLE 2
Mean Ct, reproducibility and detectability of each
standard analyzed in the SSIII-Tfi RT-qPCR assay.

| Std Conc¹ | Ct² | SD³ | # Pos⁴ |
|---|---|---|---|
| 6 | 17.2 | 0.03 | 10 |
| 5 | 20.7 | 0.07 | 10 |
| 4 | 24.2 | 0.03 | 10 |
| 3 | 27.9 | 0.04 | 10 |
| 2 | 30.9 | 0.09 | 10 |
| 1 | 33.6 | 0.13 | 10 |
| 0 | 35.3 | 0.21 | 8 |

¹Log 10 cp/μL,
²mean,
³reproducibility,
⁴number of replicates detected

### Example 2

This Example demonstrates an embodiment using Superscript III-Amplitaq360 RT-qPCR. Reverse transcriptase: Superscript III (SSIII) supplied by Invitrogen, DNA polymerase: Thermus aquaticus polymerase (AmpTaq360) supplied by Invitrogen Final reaction reagent concentrations: 50 mM Tris (pH 8.3), 75 mM KCl, 200 nM dNTP, 200 nM forward primer (5′-CCCCTGTGAGGAACTACTGT-3′), 400 nM reverse primer (5′-ACCACTATGGCTCTCCCG-3′), 200 nM Atto633-conjugated probe (5′-Atto633-AGCCATGGCGTTAGTATGAGTGTCG-IAbRQSp-3′), 2.5 mM MgCl₂, 0.2 mg/mL BSA, 3 mM DTT, 50 U SSIII, and 1.25 U AmpliTaq360 polymerase.

A 1.67× concentrated master mix is prepared and 6 μL of this master mix is added to 4 μL of RNA standard diluted in 10 mM Tris pH 7.5 containing 10 ng/μL carrier RNA to final concentrations ranging from 1×10⁰-1×10⁶ copies/μL. Ten replicates of each standard were tested. Temperature cycling parameters on the LIGHTCYCLER® 480 (Roche Life Sciences) instrument were 55° C. for 15 min, 95° C. for 3 min, 50 cycles of a) 95° C. for 5 sec followed by b) 60° C. for 30 sec. Fluorescence was only monitored over the last 40 cycles.

Data analysis—Raw fluorescence values from the LIGHTCYCLER®; 480 (Roche Life Sciences) instrument were used to fit amplification curves using the R qPCR package (freeware statistical analysis program). Ct values for each replicate were assigned by determining the 2^{nd} derivative maximum value for a 5-parameter sigmoidal fit line. Average Ct values for each standard were plotted against the Log 10 concentration for each standard and the data were fit with a linear regression line. The slope of the line was used to determine reaction efficiency (efficiency=10^{(-1/slope)}−1) and the back-calculated concentrations of each standard replicate were used to estimate sample reproducibility (standard deviation) of each standard dilution.

Raw fluorescence values from the LIGHTCYCLER® 480 (Roche Life Sciences) instrument were used to fit amplification curves using the R qpcR package (freeware statistical analysis program). Ct values for each replicate were assigned by determining the 2^{nd} derivative maximum value for a 5-parameter sigmoidal fit line. Average Ct values for each standard were plotted against the Log 10 concentration for each standard and the data were fit with a linear regression line. The slope of the line was used to determine reaction efficiency (efficiency=10^{(-1/slope)}−1) and the back-calculated concentrations of each standard replicate were used to estimate sample reproducibility (standard deviation) of each standard dilution.

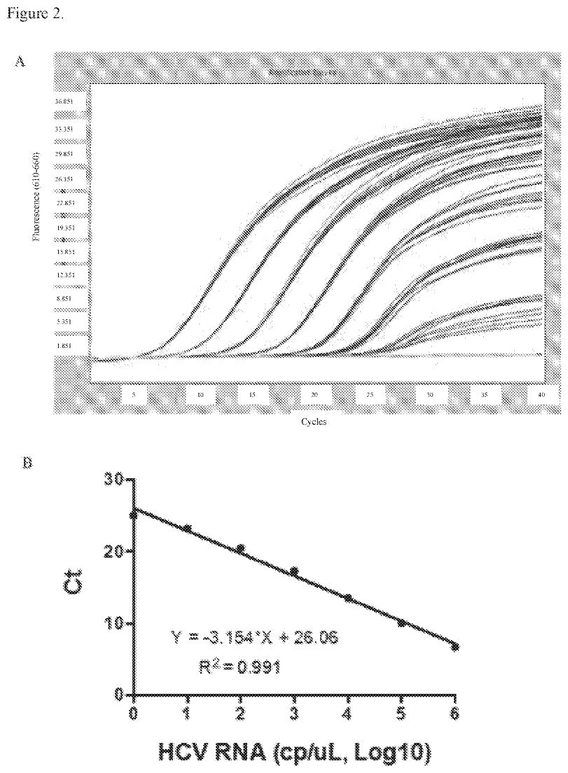

For validation of this assay, 10 replicates of each standard (1×10⁰-1× 10⁶ cp/μL) were analyzed on the LIGHTCYCLER® 480 (Roche Life Sciences) instrument using the method described above, amplification curves analyzed using the R qpcR package, standard curves generated, and assay performance was evaluated, as described above. FIG. 2 depicts amplification curves and standard curves for the Superscript III-AmpTaq360 RT-qPCR performance test. The equation of the linear regression line is included in the standard curve graph.

Ten replicates of each standard dilution were analyzed using the SSIII-AmpTaq360 RT-qPCR assay and the amplification curves for the test are depicted in FIG. 2. The mean Ct values for each standard were plotted against standard RNA concentration and the data were fit with a linear regression line with the following equation: Y=−3.154×+26.06 (FIG. 2). This slope corresponds with a reaction efficiency of 107.5%. All 10 replicates of the 4×10′-4×10⁶ cp/reaction standards (100% detection) and 9 replicates of the 4×10⁰ cp/reaction standard (90% detection) were detected (Table 3). While the assay reproducibility decreased (represented by an increasing variability) with decreasing concentration of HCV RNA standard, the average reproducibility across all standards was 0.05 Log 10 (Table 3).

TABLE 3
Mean Ct, reproducibility and detectability of each
standard analyzed in the SSIII-Tfi RT-qPCR assay.

| Std Conc¹ | Ct² | SD³ | # Pos⁴ |
|---|---|---|---|
| 6 | 16.7 | 0.02 | 10 |
| 5 | 20.0 | 0.01 | 10 |
| 4 | 23.5 | 0.01 | 10 |
| 3 | 27.3 | 0.03 | 10 |
| 2 | 30.5 | 0.05 | 10 |
| 1 | 33.2 | 0.11 | 10 |
| 0 | 35.0 | 0.12 | 9 |

¹Log 10 cp/μL,
²mean,
³reproducibility,
⁴number of replicates detected

### Example 3

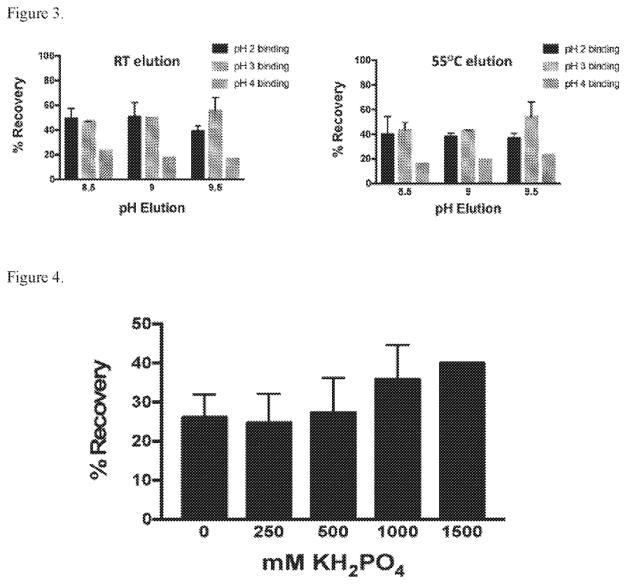

This Example demonstrates use of a HfO₂ coated surface for purification of RNA. In particular, this Example demonstrates use of a solution containing Hepatitis C RNA as described above added to a silicon oxide blank having a surface coating of hafnium oxide. FIG. 3 depicts the extraction of RNA from an RNA spiked buffer at different binding pH, elution pH and elution temperature. The percentage recovery across the ranges and temperatures for the pH values of 2 to below 4 was in excess of 40%, demonstrating sufficient recoveries of purified nucleic acids to allow for sensitive detection of pathogen nucleic acids in blood or other biological specimens. FIG. 4 depicts titration of KH₂PO₄ concentration in wash buffer during extraction of RNA from plasma, indicating that the presence of kosmotropic KH₂PO₄ is effective as recovery of purified RNA exceeds 40% at 1500 mM KH₂PO₄.

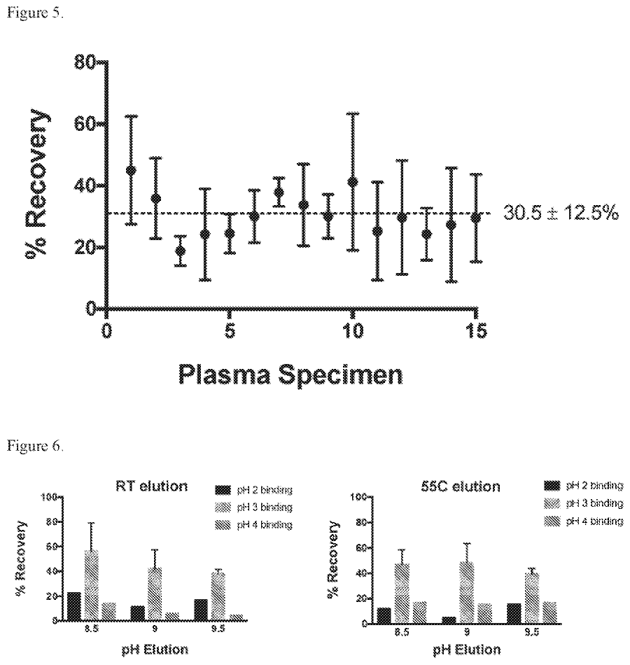

FIG. 5 depicts Extraction of RNA from 15 plasma specimens obtained from 15 different donors, with average recovery at 30.5% and variance from the mean at 12.5%, indicating that recovery of pathogen RNA is accomplished across a wide range of plasma specimens.

### Example 4

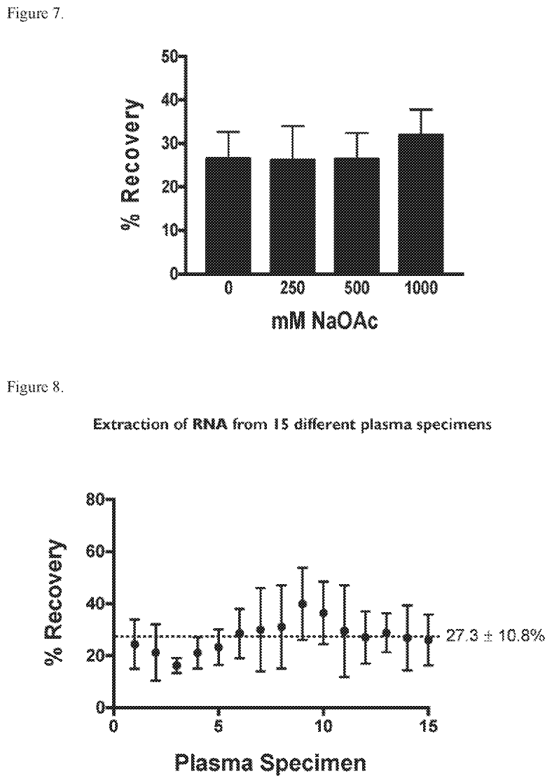

This Example demonstrates extraction and purification of RNA from buffer on an Al₂O₃ coated surface. A solution containing Hepatitis C RNA was prepared as described above and added to a silicon oxide blanket having a surface coating of aluminum oxide. FIG. 6 depicts the extraction of RNA from an RNA spiked buffer at different binding pH, elution pH and elution temperature. The percentage recovery across the ranges and temperatures for the pH of 3 was in excess of 40%, demonstrating sufficient recoveries of purified nucleic acids to allow for sensitive detection of pathogen nucleic acids in blood or other biological specimens. FIG. 7 depicts titration of NaOAc concentration in wash buffer during extraction of RNA from plasma, indicating that the presence of kosmotropic NaOAc is effective as recovery of purified RNA exceeds 30% at 1000 mM NaOAc. FIG. 8 depicts extraction of RNA from 15 plasma specimens obtained from 15 different donors, with average recovery at 27.3% and variance from the mean at 10.8%, indicating that recovery of pathogen RNA is accomplished across a wide range plasma specimens.

### Example 5

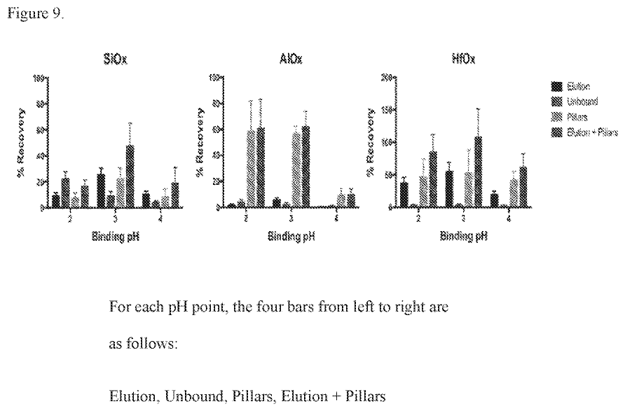

This Example demonstrates extraction and purification of RNA in plasma from micropillar structures with a binding buffer pH of from 2 to 4. In more detail, a solution containing 1×10⁵ Hepatitis C RNA in plasma was prepared and added to reaction surfaces having pillars of silicon oxide, hafnium oxide and aluminum oxide. A pH 4 wash buffer having a range of kosmotropic salts (Si—none, Hf—K₂HPO₄, Al—NaOAc) was used, followed by a basic elution buffer at 55° C. (Si—pH 9.5, Hf—9.5, Al—8.5), with other conditions as described above. Note, the pillars were removed from the reaction surface then treated with the elution buffer, and unbound fractions purified from the elution buffer with a Qiagen viral RNA purification kit. Elution and pillar-bound RNA was analyzed by an SSIII-AT360 assay. FIG. 9 shows the percentage of recovery of Hepatitis C RNA from the pillars, elution, and pillars+elution buffer, showing the advantages of using kosmotropic salts and pillar structures to improve adhesion to the reactor surface and increased nucleic acid recovery. In FIG. 9, for each of the four pH values at each pH value (2, 3 and 4), the columns in the graph are from left to right: Elution, Unbound, Pillars, Elution+Pillars.

### Example 6

This Example demonstrates extraction and purification of RNA from buffer on a pillar surface. The above protocol from Example 5 was performed using pillars scraped from a silicon oxide coated chip with plasma standards having concentrations of Hepatitis C RNA ranging from ranging from 1×10⁰-1×10⁶ copies/μL, and using binding buffer at pH 2.5 and a wash buffer at pH 2.3 Purification and positive detection of RNA in plasma was achieved at concentrations ranging from 10 copies to 1×10⁶ per μL. As shown in Table 4 below, average recoveries even for extremely low concentrations of RNA were at or near 50% and above, approaching the recoveries obtained by commercially available kits.

TABLE 4

| Std Conc¹ | % recovery² | SD³ | % CV |
|---|---|---|---|
| 6 | 66 | 18 | 27 |
| 5 | 57 | 21 | 37 |
| 4 | 51 | 20 | 39 |
| 3 | 48 | 16 | 33 |
| 2 | 49 | 17 | 35 |
| 1 | 69 | 75 | 109 |

¹Log 10 cp/μL,
²mean,
³standard deviation,

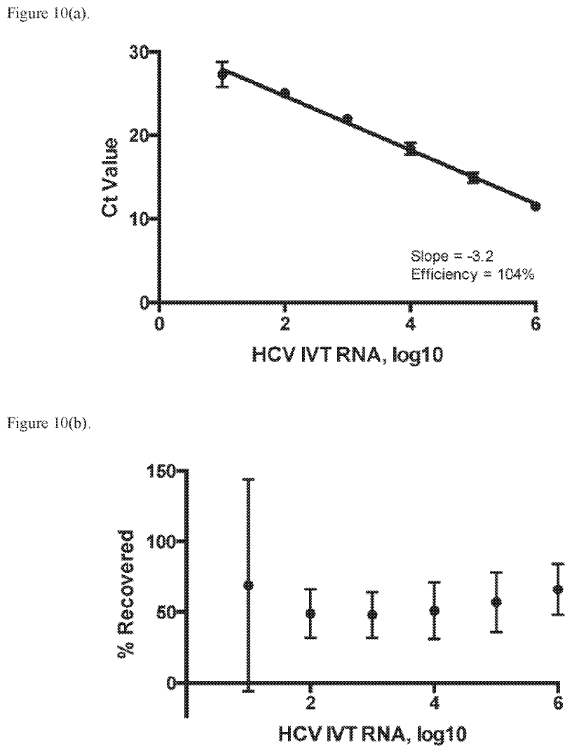

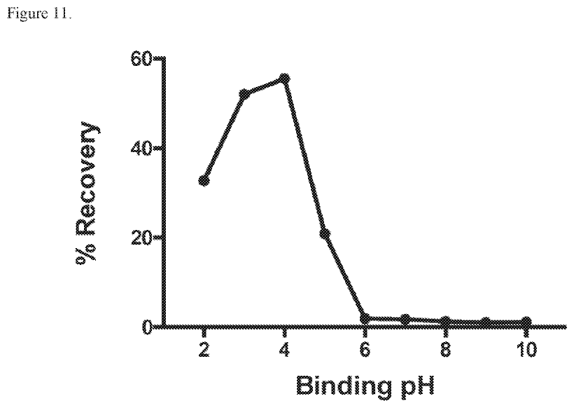

FIG. 10(a) shows the Cycle threshold (Ct) of well below 30 for all concentrations, indicating robust recovery of nucleic acids from the plasma, while FIG. 10(b) shows that the percentage recoveries averaged near 50% or higher.

### Example 7

This Example demonstrates recovery of RNA in distinct reaction conditions.

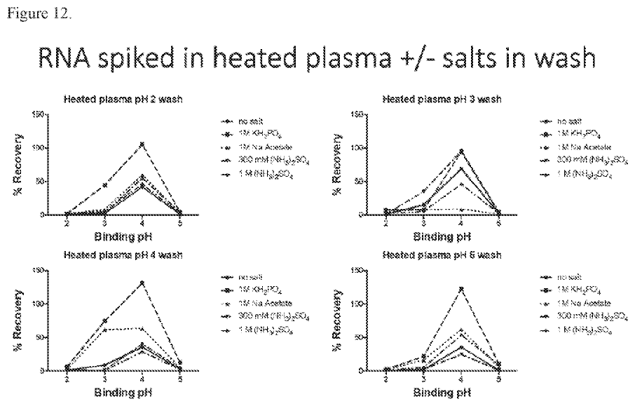

Data summarized in FIG. 11 show recovery of RNA (1×10⁵ cp/μL) spiked in binding buffer of pH 2-10 in plasma at different pH values, salts and temperatures. In particular, as shown in FIG. 11 RNA spiked in 20 mM buffer (pH 2-10) was incubated with scraped silicon oxide-coated pillars for 10 min, washed 2× with binding buffer and then the amount of RNA bound to pillars was determined. However, and without intending to be constrained by any particular approach, the data demonstrate that maximal binding of RNA spiked in buffer occurs at pH 3-4.

FIGS. 12 and 13 summarize recovery of RNA (1×10⁵ cp/μL) using binding buffers over a pH 2-5 range. RNA was spiked into binding buffer (containing 300 mM (NH₃)₂SO₄) and plasma that was heated for 10 min (FIG. 12) at 55° C. (to model heat-based lysis) or unheated plasma with proteinase (to model a chemical-based lysis; FIG. 13) and that mixture was incubated with scraped silicon oxide-coated pillars for 10 min. For FIG. 12 the pillars were washed 1× with wash buffer (20 mM pH 2, 3, 4 or 5) with or without (control) kosmotropic salts and then washed 1× with wash buffer without salts. The amount of RNA bound to pillars was then analyzed by RT-qPCR. As shown addition of kosmotropic salts significantly increased recovery.

For FIG. 13, RNA was spiked into normal human plasma and this was diluted with binding buffer (pH 2-5) containing 300 mM (NH₃)₂SO₄, DTT (1 mM, final cone), RNasin (1:20 dilution) and proteinase K (0.25 mg/ml final cone). This was added to pillars scraped from silica oxide-coated chips and incubated for 10 min. The plasma/binding buffer was removed and pillars were washed with 1) 20 mM NaAcetate pH 4 containing 1 M KH₂PO₄ and then 2) 20 mM NaAcetate pH 4. Bound RNA was then quantified using the HCV RNA RT-qPCR assay. The results are shown in FIG. 13.

### Example 8

This Example demonstrates extraction and amplification of RNA by capillary flow, lysis of HCV particles as quantified by a device sold under the trade name LIGHTCYCLER®, 480 for analysis of RNA, and use of an internal RNA control to normalize results.

To obtain the data shown in FIG. 17, HCV RNA purified from HCV cell culture supernatant was spiked into normal human plasma and this mixture was added to binding buffer (HCl/KCl buffer pH 2.5 with 300 mM (NH₃)₂SO₄, 1 mM DTT, 10 ng/μL carrier RNA, RNasin (1:20 dilution)) and added to a chip. The chip comprised a reservoir, a channel leading to a chamber with micropillars for RNA binding, and second channel that comprising a second micropillar array (a capillary pump). RNA/binding buffer flowed across the extraction chamber. The chip was washed via capillary flow with wash buffer 1 (Sodium Acetate pH 4 with 1 M KH₂PO₄) and then wash buffer 2 (Sodium Acetate pH 4). Pillars were scraped from the chip and HCV RNA was quantified using a LIGHTCYCLER. Percent recovery shown on the Y axis represents the amount of RNA detected on-pillars compared the amount of RNA spiked into binding buffer.

FIG. 15 depicts results obtained from lysis of viral particles and analysis of viral RNA to measure percentage of particles lysed. FIG. 15A) HCV viral particles obtained from cell culture were suspended in PBS, added to binding buffer (HCl/KCl buffer pH 2.5 with 300 Mm (NH₃)₂SO₄, 1 mM DTT, 10 ng/μL carrier RNA, RNasin (1:20 dilution)) containing 0.1-0.6% SDS and incubated with scraped pillars. Pillars were washed with wash buffer 1 (Sodium Acetate pH 4 with 1 M KH₂PO₄) and then wash buffer 2 (Sodium Acetate pH 4). Amount of HCV bound to RNA was quantified on a LIGHTCYCLER. HCV viral particles obtained from cell culture were suspended in PBS, added to binding buffer (HCl/KCl buffer pH 2.5 with 300 mM (NH₃)₂SO₄, 1 mM DTT, 10 ng/μL carrier RNA, RNasin (1:20 dilution)) containing 0.1-0.6% SDS and incubated with scraped pillars at 55° C. for 5 min (FIG. 15B) or 75° C. for 2 or 5 min (FIG. 15C). Pillars were washed with wash buffer 1 (Sodium Acetate pH 4 with 1 M KH₂PO₄) and then wash buffer 2 (Sodium Acetate pH 4). Amount of HCV bound to RNA was quantified on the LIGHTCYCLER.

FIG. 16 depicts use of an internal RNA control for comparison with input RNA. To obtain the data depicted on the graph, 4×10⁴ copies of in vitro transcribed HCV and Physcomitrella patens (spreading earthmoss, internal control) RNA was amplified in the same RT-qPCR reaction and fluorescence was monitored across 50 cycles. The average fluorescence of the first ten cycles was used to normalize the fluorescence in cycle 10-50 (first 10 cycles not represented in figure).

FIG. 20 depicts on-chip amplification of HCV RNA. The chip comprised a reaction chamber without pillars mounted on a printed circuit board. The reagents were pipetted onto the chip and an integrated controlled by computer controlled temperature. Fluorescence was monitored using a fluorescent microscope FIG. 20, (left panel) shows amplification curves for 1×10⁶-1×10 in vitro transcribed an HCV in vitro transcribed RNA standard. Final reagent concentrations are as follows: 10 mM Tris pH 8.4, 75 mM KC 1, 2.5 mM MgCl₂, 200 uM dNTP, 200 nM forward primer, 200 nM fluorescently labeled probe, 400 nM reverse primer, 10 ng/μL carrier RNA, 0.2 mg/mL BSA, 3 mM DTI, 50U SuperScript III and 5U AmpTaq360. Cycling conditions are as follows: 5 min at 55° C. 3 min at 95° C., 50 cycles of 5 sec at 95° C. followed by 10 sec at 60° C. Fluorescence was measured at the end of the RT step and after each cycle. The post-RT fluorescence was subtracted from fluorescence at each cycle and then the average fluorescence of the first 10 cycles was used to normalize the fluorescence values of cycles 11-50. The fluorescence from the first 10 cycles is not displayed on graph. Curves were fit using the 5-parameter model in the qPCR program in R. FIG. 20, (right panel) shows Ct values that were identified by determining the second derivative maximum of the fit line and plotted against standard concentration. Linear regression was used to fit a line and the slope and fit of the line is denoted on the graph. Thus, FIG. 20 demonstrates performance of an efficient and sensitive assay on-chip in the presence of silica and using integrated heaters.

### Example 9

This Example expands the examples above, and demonstrates sensitive (4 copies/reaction) RT-qPCR and qPCR assays detecting HCV, HIV, Zika, HPV 16, and HPV 18 on a benchtop real-time PCR instrument.

In more detail, in this Example, we developed rapid and sensitive NAT for a number of RNA and DNA viruses on the same silicon microchip platform. We first developed sensitive (limit of detection (LOD), 4 copies/reaction) one-step RT-qPCR and qPCR assays detecting HCV. HIV, Zika. HPV 16, and HPV 18 on a benchtop real-time PCR instrument. The same novel silicon microchip design with an etched meandering microreactor, integrated aluminum heaters, thermal insulation trenches and microfluidic channels for delivery of reagents was used for all on-chip experiments in this Example following demonstration of precise and localized heating of the microreactor using melting temperature analysis. Following minimal optimization of reaction conditions, the bench-scale one-step RT-qPCR and qPCR assays were successfully transferred to 1.3 μL silicon microreactors with reaction times of 25 min and no effect on the LOD. Also, the reproducibility and reaction efficiencies of the bench scale and on-chip assays was similar. Taken together, these results demonstrate that rapid and sensitive detection of multiple viruses on the same silicon microchip platform is feasible. Further development of this technology, coupled with silicon microchip-based nucleic acid extraction solutions, could potentially shift viral nucleic acid detection and diagnosis from centralized clinical laboratories to the POC.

The following materials and methods were used to obtain the results described in this Example.

Oligonucleotides and Standards

All primers and hydrolysis probes were designed using PrimerBlast (NCBI) and publically-available sequences (Genmed, NCBI) and synthesized by IDT technologies (Table 1). In vitro transcribed (IVT) viral RNA was purchased (Amsbio, Mass.) and used as a standard for each RNA target (Table 2). For the DNA standards, linearized pHPV-16 plasmid DNA (clone 45113D, ATCC, Virginia) and pHPV-18 plasmid DNA (clone 45152D, ATCC, Virginia) were used as standards.

Bench Scale Assay Development of RNA Viruses

All bench scale RT-qPCR assays were developed using a LightCycler480 instrument (Roche, Switzerland) and IVT RNA for each target was used as a standard. Amplification was performed using 10 μl, reactions containing 50 mM TRIS pH 8.3, 75 mM KCl, 200 μM dNTP Mix (Invitrogen, California), 200 nM forward primer (IDT Technologies, Iowa), 400 nM reverse primer (IDT Technologies, Iowa), 200 nM hydrolysis probe (IDT Technologies, Iowa), 0.2 mg/mL BSA (Thermo Fisher, Massachusetts), 3 mM DTT (Thermo Fisher, Massachusetts), 50 units SuperScript III (Thermo Fisher, Massachusetts) and 1.25 units of AmpliTaq360 polymerase (Thermo Fisher, Massachusetts). The HCV and HIV assays contained 2.5 mM MgCl₂ (Invitrogen, California) while the Zika assay contained 3 mM MgCl₂. The HCV assay cycling conditions included a 15 minute RT step at 55° C., 3 minute initial denaturation step at 95° C., and 50 cycles of 10 second denaturation at 95° C. and 30 second amplification at 60° C. for a total assay time of 70 minutes. Both the HIV and Zika assays used cycling conditions that included a 5 minute RT step at 55° C., 3 minute initial denaturation step at 95° C., and 50 cycles of 5 second denaturation at 95° C. and 10 second amplification at 60° C. for a total assay time of 51 minutes. The standard concentrations used in experiments ranged from 4×10⁶-4×10⁰ copies per reaction.

Bench Scale Assay Development of DNA Viruses

Bench scale assays were developed for DNA viruses HPV 16 and HPV 18. Amplification of the HPV targets was performed using 10 μl reactions containing 50 mM TRIS pH 8.3, 75 mM KCl, 200 μM dNTP Mix (Invitrogen, California), 200 nM forward primer (IDT Technologies, Iowa), 400 nM reverse primer (IDT Technologies, Iowa), 200 nM hydrolysis probe (IDT Technologies, Iowa), 0.2 mg/mL BSA (Thermo Fisher, Massachusetts), 3 mM DTT (Thermo Fisher, Massachusetts), and 1.25 units of AmpliTaq360 polymerase (Thermo Fisher, Massachusetts). The standard concentrations used in experiments ranged from 4×10⁶-4×10⁰ copies per reaction. The HPV 16 assay contained 1.5 mM MgCl₂ (Invitrogen, California) while the HPV 18 assay contained 4.5 mM MgCl₂. The cycling conditions for the HPV 16 assay included a 3 minute initial denaturation step at 95° C., and 50 cycles of 10 second denaturation at 95° C. and 30 second amplification at 60° C. for a total assay time of 67 minutes. The HPV 18 assay utilized the same protocol but with 30 second amplification at 62° C. instead of 60° C.

Bench Scale Data Analysis

For bench scale assays, Ct values were determined using the Lightcycler 480 software. Although 50 cycles were performed for each assay, the reaction fluorescence was not monitored during the first ten cycles. Therefore, at the end of each run, 10 cycles were added to the Ct values calculated using the LightCycler480 software. Average Ct values for each standard were plotted against the Log 10 standard concentrations and linear regression line was determined. The slope of the line was used to determine the reaction efficiency (efficiency=(10^{(-1/slope)}−1) and the back-calculated concentrations of each standard replicate were used to estimate sample reproducibility (standard deviation) of each standard dilution.

Chip Fabrication and Characterization

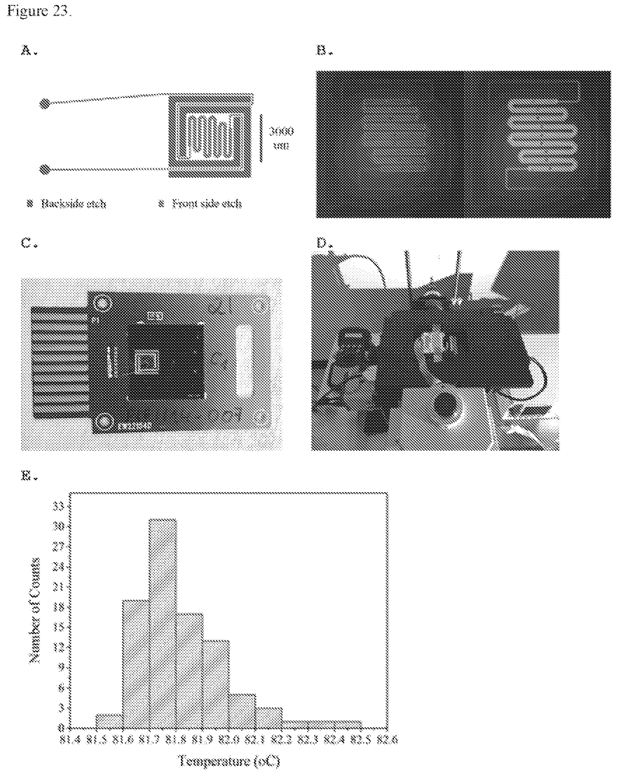

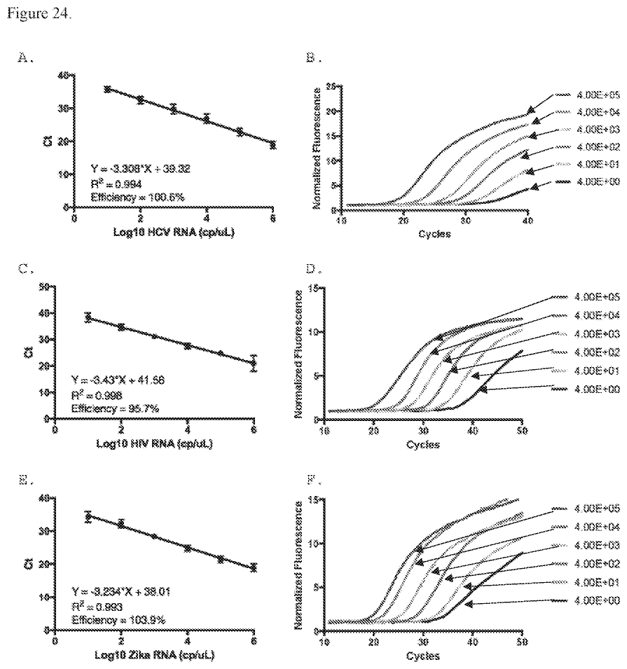

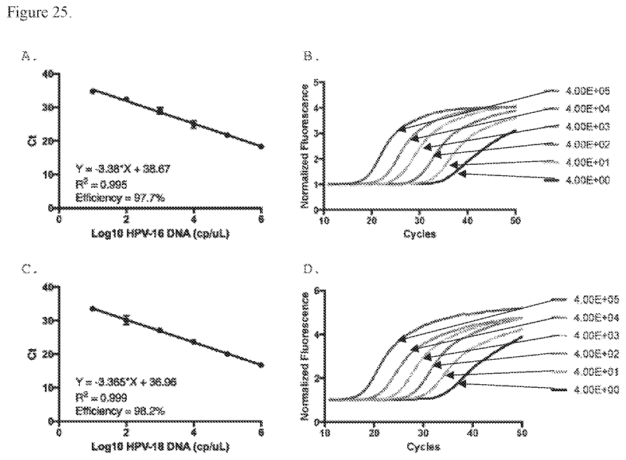

The PCR reactor was fabricated using silicon-glass technology. Details of the fabrication have been reported previously⁶ and are briefly summarized here (FIG. 23). First, the fluidic structures and the thermal insulation trenches were sculpted on the front side of the silicon by standard lithography and deep reactive ion etching. Then, a Pyrex wafer was anodically bonded to the silicon to seal the channels. A backside etch was performed to open access holes and to etch the thermal insulation trenches completely through the silicon. Finally, the heater, consisting of a meandering aluminum resistor, was deposited on the backside of the silicon and electrically insulated from it by a thin silicon oxide layer. The PCR chamber is a long, meandering silicon microchannel with a width of 200 μm, a depth of about 220-230 μm, and a resulting volume of 1.3 μL. The meandering shape helps to compensate for thermal losses and avoids trapping air bubbles during filling. The inlet/outlet ports have a diameter of 750 μm, allowing a tight fit of standard pipette tips which creates a small pressure when loading and contributes to regular filling of the cavity. The temperature is measured by a resistance temperature detector (RTD) fabricated in the same aluminum layer as the heater. In addition, a thermistor is placed on the printed circuit board (PCB) to monitor the temperature of the bulk of the chip. The fabricated microreactor was mounted on a simple, custom-made PCB shown in FIG. 23 and contacts on the chip were wire-bonded to the PCB contacts. The PCB was in turn inserted in a holder connected to a dedicated instrument for temperature control, which was built in-house. The holder was placed on the stage of an inverted fluorescence microscope (Olympus IX-73) equipped with a CMOS camera (Orca Flash 4.0, Hamamatsu. Japan) and fluorescent light source (X-Cite exacte. Excelitas Technologies). A script was written in LabVIEW (National Instruments) to control temperature and acquire fluorescent images after each cycle of PCR amplification. Temperature uniformity of the microreactor was assessed by melting curve analysis using DNA fragments with known melting temperatures (sequences available upon request). Fluorescence images were taken at regular time intervals while increasing the temperature with a constant ramp rate. Melting temperature was determined as the point at which the second derivative of the fluorescence intensity reached a maximum.

Transfer of Bench Scale Assays to Silicon Microchip Reactor

The optimized bench scale assays for both RNA and DNA targets were then transferred to the silicon microreactor with some modifications. All reagent concentrations were the same as the bench scale assay except the AmpliTaq360 concentration was increased to 5 units per reaction for all targets on-chip. The same standards used to develop each bench scale assay were tested on-chip with a range of 4×10⁰-4×10⁵ copies per reaction. Reactions were loaded into the reaction chamber and amplified according to the following cycling conditions. The HCV assay included a 5 minute RT step at 55° C., 3 minute initial denaturation step at 95° C., and 40 cycles of 5 second denaturation at 95° C. and 10 second amplification at 60° C. for a total assay time of 24.8 minutes. The cycling conditions for both the HIV and Zika assays included a 2.5 minute RT step at 55° C., 1.5 minute initial denaturation step at 95° C., and 50 cycles of 5 second denaturation at 95° C. and 10 second amplification at 60° C. for a total assay time of 25 minutes. The HPV 16 and HPV 18 assays included a 1.5 minute initial denaturation step at 95° C. and 50 cycles of 5 second denaturation at 95° C. and 10 second amplification at either 60° C. (HPV 16) or 62° C. (HPV 18) for a total assay time of 22.5 minutes. Between runs, chips were cleaned by incubating the microreactor in 10% bleach at 95° C. for 5 min followed by one wash with water at 95° C. for 5 mins and then two additional room temperature water washes.

On-Chip Image Analysis

During the RT-qPCR on-chip program, images of the reaction chamber were captured using the inverted fluorescence microscope. The first image was captured at the start of the run, the second image was captured at the end of the RT step, and the remaining images were captured at the end of each amplification cycle. All images were then analyzed using ImageJ analysis software (NIH). The reaction chamber was divided into 9 sections with each section encompassing a meander (FIG. 23). In order to ensure the analysis was identical between chips, the 9-box configuration was saved, imported, and aligned to each series of chip images. The mean fluorescence intensity (MFI) was then calculated across all images for each of the nine meanders using the multi-measure function in the software package. The chip background MFI (post-RT, RNA; start of run, DNA) was subtracted from all subsequent MFI values. The resultant chip-normalized MFI values were then divided by the average MFI across the first 10 cycles (assay background). These normalized fluorescence values were used to fit amplification curves and determine the Ct of each standard.

On-Chip Ct Determination

Ct values were determined for each standard replicate using the qPCR package in the R Studio software (RStudio, Boston, Mass.) by calculating the 2^{nd} derivative maximum for a 5-parameter sigmoidal fit line. Average Ct values for each standard were plotted against Log 10 standard concentrations and a linear regression line was determined. The slope of the line was used to determine reaction efficiency (efficiency=(10^{(-1/slope)}−1) and the back-calculated concentrations of each standard replicate were used to estimate sample reproducibility (standard deviation) of each standard dilution.

The following results were obtained using the materials and methods of this Example as described above.

Bench Scale RNA Assays

In order to test the performance of each bench scale RNA assay, three independent experiments were performed in which IVT RNA standard dilution series (4×10⁶-4×10⁰ copies/reaction) were tested. For all targets, each standard concentration was detected in each independent experiment suggesting an assay sensitivity of at least 4 cp/reaction for each target. The mean Ct values calculated across the three independent experiments were plotted against the corresponding Log 10 RNA concentrations for each target. The overall efficiency of each assay, derived from the slope of the linear regression line, was 107.8% for HCV, 109.8% for HIV, and 98.8% for Zika. In order to assess assay variability, the Ct values of each standard replicate was used to determine a back-calculated RNA concentration from the average efficiency curve across the three replicates. The average variability of all RNA targets was less than 0.5 Log 10 (HCV, 0.05 Log 10; HIV, 0.35 Log 10; and Zika, 0.10 Log 10).

Bench Scale DNA Assays

The performance of each bench scale assay was assessed using three independent experiments were performed in which plasmid DNA standard dilution series (4×10⁶-4×10⁰ copies/reaction) were tested. For both HPV 16 and HPV 18, each standard concentration was detected in each independent experiment indicating an assay sensitivity of at least 4 cp/reaction for each target. The mean Ct values calculated across the three independent experiments were plotted in the same way as the RNA bench scale assays and the resulting reaction efficiencies for HPV 16 and HPV 18 were 108.9% and 100.9%, respectively. The average variability, based on back-calculated concentrations, of both DNA targets was less than 0.5 Log 10 (HPV 16, 0.16 Log 10; and HPV 18, 0.08 Log 10).

Characterization of the PCR Microchip

Prior to on-chip assay development, a series of tests were performed. After mounting the chip on the PCB, the RTDs were calibrated in an oven to ensure correct measurement of the temperature during PCR. The temperature values and uniformity were further verified by melting curve analysis using DNA fragments with known melting temperatures close to the annealing temperature of the PCR primers (Tm=60° C. and 70° C.) and to the denaturation temperature (Tm=80° C.). At 80° C., temperature uniformity across the microreactor was 0.7° C. and the measured temperature was correct within 0.5° C. The ramp time from 60° C. to 95° C. was 2 s at a heater current of 0.1 A. The cooling time from 95° C. to 60° C. was 4 s and is fixed by the thermal design. During temperature cycling, the bulk temperature of the chip surrounding the PCR microreactor did not exceed 50° C., due to the thermal insulation trenches (FIG. 3).

On-Chip RNA Assays

In order to assess the performance of the on-chip RNA assays, three independent experiments were performed during which each IVT RNA standard across the standard range (4×10⁵-4×10⁰ copies/reaction) was tested on a microchip. All standard replicates of each target were detected, suggesting an assay sensitivity of at least 4 cp/reaction. The mean Ct values calculated across the three replicates were plotted in the same way as the bench scale assays resulting in reaction efficiencies of 100.6%, 95.7%, and 103.9% for HCV, HIV, and Zika respectively. The average variability, based on back-calculated concentrations, across all standard concentrations of HCV, HIV, and Zika RNA were 0.35 Log 10, 0.41 Log 10, and 0.32 Log 10, respectively.

On-Chip DNA Assays

In order to assess the performance of the on-chip DNA assays, three independent experiments were performed during which each plasmid DNA standard across the standard range (4×10⁵-4×10⁰ copies/reaction) was tested on a microchip. All standard replicates of each target were detected, indicating an assay sensitivity of at least 4 cp/reaction. The efficiencies for HPV 16 and HPV 18 were 97.7% and 98.2% respectively. The average reproducibility, based on back-calculated concentrations, across all standard concentrations was 0.18 Log 10 for HPV 16 and 0.17 Log 10 for HPV 18.

It will be recognized that the foregoing results support the feasibility of detecting viral nucleic acids using novel RT-qPCR and qPCR assays and microreactors on silicon microchip. Bench scale RT-qPCR assays for HCV, HIV, and ZIKV and qPCR assays for HPV 16 and HPV 18 with assay sensitivities of 4 cp/reaction were developed using a standard bench scale real-time PCR instrument. PCR silicon microchips with 1.3 μL microreactors and rapid and consistent temperature cycling were designed and manufactured. Finally, these assays were successfully transferred to the developed PCR silicon microchips with little optimization and no loss in sensitivity (4 cp/reaction) and reproducibility (<0.5 Log 10). These results demonstrate that rapid and sensitive amplification of viral nucleic acids in a 1.3 μL microreactor is feasible and supports nucleic acid-based diagnostic devices using scalable silicon microchip technology.

Previously available POC serologic tests for diagnosis of acute viral infection suffer from decreased clinical sensitivity since delayed antibody production limits the sensitivity of immunoassays in the first week of illness. Also, once detectable, persistence of antibodies complicates the identification of current versus past infection. While diagnostic NAT are considered the gold standard for detection of viral infections, they require large, expensive equipment and skilled operators. As a result, prior to the present disclosure, viral NAT must be performed in centralized clinical laboratories which leads to a long time-to-result. All of these can increase diagnostic uncertainty at the POC leading to inappropriate antimicrobial use, a significant public health concern. Providing not only sensitive, but fast, POC viral NAT may overcome these limitations and enable clinicians to more accurately diagnose viral infections at the point-of-need. In this regard, the presently demonstrated five on-chip assays provide reproducible and sensitive detection of HCV. HIV, Zika. HPV-16, and HPV-18. Additionally, the silicon microchip technology utilized in this study allows for rapid and accurate thermal cycling and a shortened time-to-result. Indeed, our viral nucleic acid assays were performed in 25 minutes, significantly shorter than the 51 minute bench scale assay. Further optimization of the assays and silicon microchip design can be achieved given the benefit of this disclosure, and is expected to lead to shorter cycle and total reaction times. For instance, the HCV assay was able to be performed in 40 cycles with an increase in the length of the RT step time while the other assays needed 50 cycles to maintain sensitivity and efficiency with a shorter RT step. This suggests that cycling time optimization for each target is necessary for sensitive and efficient assays in silicon microreactors. Utilizing these assays on silicon microchip-based diagnostic devices may significantly decrease the time-to-result of molecular POC diagnostic devices.

The flexibility and scalability of silicon microchips allows for integration with other fluidic solutions. For instance, these microreactors may be coupled with microfluidic plasma separation and nucleic acid extraction solutions resulting in a POC diagnostic device that accepts low volume (<50 μL) whole blood specimens without the need of sample preparation other than blood collection. The standard concentration range used in this study was based on concentrations of viral nucleic acids in body fluids during acute and chronic viral infection. Using the standard concentration range in this study and a hypothetical device containing coupled microfluidic plasma separation and nucleic acid extraction, one would be able to detect down to 640 cp/mL plasma assuming a 50 μl, blood specimen (25 μl, of plasma), 50% on-chip recovery of plasma, and 50% on-chip nucleic acid extraction yield. This detection limit would allow for sensitive diagnosis of most acute and chronic viral infections. For example, non-human primate studies have demonstrated peak ZIKV RNA levels in plasma (>10⁵ cp/mL plasma) for the first week of acute infection at levels significantly higher than our theoretical limit of detection. Additionally, this theoretical lower limit of detection would allow for detection of >99.5% of chronic HCV infections. Therefore, incorporation of these ultralow volume reactions in silicon microchip-based molecular diagnostics can result in sufficient sensitivity to diagnose acute and chronic viral infections.

It will be recognized from the foregoing description of this Example that silicon microchips with an etched meandering microreactor, patterned aluminum heaters, resistance temperature detectors, thermal insulation trenches and microfluidic channels for delivery of reagents were produced and precise heating of only the microreactor was demonstrated. Then, the benchscale viral RNA and DNA assays were successfully transferred to the 1.3 μL silicon microreactor. The on-chip reaction sensitivity and efficiency were similar to the bench scale assays but importantly, the 25 min or less reaction time was significantly shorter than the benchscale assays. This disclosure of this Example can be combined with silicon microchip-based nucleic acid extraction methods discussed above, with the intent to shift viral nucleic acid detection and diagnosis from advanced clinical laboratories to the POC.

In conclusion, this Example and those above it provide silicon microchip molecular diagnostic devices for viral infections, and for detecting any polynucleotides. We developed bench scale PCR-based assays for detection of RNA and DNA viruses that were successfully transferred to silicon microchip microreactors with minimal optimization. This finding indicates that the silicon microchip technology can be adapted to detect a broad range of pathogens without the need for significant microchip design or assay optimization. Coupling these with on-chip plasma separation and nucleic acid extraction can provide a platform for the development of rapid and sensitive sample-to-result POC molecular diagnostic solutions that should significantly decrease assay time-to-result, thereby increasing diagnostic certainty in all clinical settings.

Although the present disclosure has been described in connection with preferred embodiments thereof, it will be appreciated by those skilled in the art that additions, deletions, modifications, and substitutions not specifically described may be made without departing from the spirit and scope of the present disclosure as defined in the appended claims.

1. A process for the purification and detection of nucleic acid amplification products, comprising:
a. delivering a sample having unpurified nucleic acids into a microfluidic region;
b. contacting the nucleic acids with a fixed surface in the microfluidic region, wherein the nucleic acids adhere to the surface;
c. washing the microfluidic region and surface with a first buffer;
d. washing the microfluidic region and surface with a second buffer, wherein the second buffer has a pH that is equal to or higher than the first buffer;
e. amplifying at least some of the nucleic acids to produce an amplification product; and
f. detecting the amplification product;
wherein the step of delivering the nucleic acids to the microfluidic region uses an adhesion solution comprising kosmotropic salts and optionally a nuclease inhibitor, and wherein the adhesion solution is free of chaotropic salts and ethanol.

2. The process of claim 1, wherein the first buffer further comprises NaCl and has a pH of from 1 to 4.5.

3. The process of claim 1, wherein the second buffer has a pH of from 1 to 4.5, and does not contain kosmotropic salts.

4. The process of claim 1, wherein the sample is a biological sample, the method further comprising lysing viruses and/or cells in the biological sample to release unpurified nucleic acids into solution as part of the step of delivering the nucleic acids into a microfluidic region.

5. The process of claim 1, wherein the step of contacting the nucleic acids with a fixed surface in the microfluidic region includes incubating nucleic acids in the microfluidic region at a temperature of between 20-80° C.

6. The process of claim 1, wherein the fixed surface comprises a metal oxide or a metal nitride or a silicon oxide or a silicon nitride.

7. The process of claim 6, wherein the metal oxide is aluminum oxide (Al₂O₃), or hafnium oxide (HfO₂),

8. The process of claim 4, wherein the step of lysing the viruses and/or cells in the biological sample comprises releasing nucleic acids by heating the biological sample in the adhesion buffer, or by exposure of the biological sample in the adhesion buffer to a chemical composition such that the lysis occurs, wherein the chemical composition optionally comprises from 0.1-1.0% SDS and/or from 0.1-0.5% NP-40 detergent.

9. The process of claim 8, wherein the biological sample is heated in the adhesion buffer in the microfluidic region.

10. A kosmotropic solution for microfluidic amplification assays, wherein the kosmotropic salt is KH₂PO₄, or (NH₄)₂SO₄, K₂SO₄, the solution optionally comprising 1-35% DMSO.

11. A system for the purification and amplification of nucleic acid sequences using the method of claim 1, comprising a microfluidic reactor with at least one fixed surface having a metal oxide or coating or silicon oxide (SiO₂) coating or silicon nitride coating, said coating consisting essentially of aluminum oxide (Al₂O₃), hafnium oxide (HfO₂), silicon nitride (Si₃N₄), or silicon oxide (SiO₂).

12. The system of claim 11, wherein the at least one surface is present on a plurality of micropillars in the microfluidic reactor.

13. The system of claim 12, wherein the plurality of micropillars have at least one of the following characteristics: i) a micropillar height of from approximately 190-200 μm; ii) a micropillar width of approximately 20 μm; a center-to-center micropillar distance of approximately 50 μm; iii) an interpillar distance of about 30 μm.

14. The system of claim 13, wherein at least some of the plurality of micropillars are in non-covalent association with polynucleotides.

15. A process for determining nucleic acids comprising:
a. contacting a biological sample comprising or suspected of comprising nucleic acids with a surface, wherein the nucleic acids if present adhere to the surface;
b. washing the adhered nucleic acids and the surface with a first buffer;
c. washing the adhered nucleic acids and the surface with a second buffer, wherein the second buffer has a pH that is equal to or higher than the first buffer;
d. amplifying at least some of the nucleic acids to produce an amplification product; and
e. detecting the amplification product;
wherein the step of contacting the nucleic acids with the surface is performed using an adhesion solution comprising kosmotropic salts and optionally a nuclease inhibitor.

16. The process of claim 15, wherein the surface comprises a metal oxide coating or silicon oxide or silicon nitride coating, said coating consisting essentially of aluminum oxide (Al₂O₃), hafnium oxide (HfO₂), silicon nitride (Si₃N₄), or silicon oxide (SiO₂).

17. The process of claim 16, wherein the surface is present on a plurality of micropillars.

18. The process of claim 17, wherein the plurality of micropillars have at least one of the following characteristics: i) a micropillar height of from approximately 190-200 μm; ii) a micropillar width of approximately 20 μm; a center-to-center micropillar distance of approximately 50 μm; iii) an interpillar distance of about 30 μm.

19. The process of claim 15, wherein the sample comprises the nucleic acid, and wherein at least 15%, and up to 40% of the nucleic acid content in the sample adheres to the surface in step a.

20. The process of claim 15, wherein at least 15% of the nucleic acid content in the sample is amplified to obtain the amplification product of step d.

21. The process of claim 19, wherein said nucleic acid content is from 35-100% of the nucleic acid content in the sample.

22. The process of claim 21, wherein said nucleic acid content is from 40% of the nucleic acid content in the sample.

23. A vessel comprising a surface comprising a metal oxide coating or silicon oxide or nitride coating, said coating consisting essentially of aluminum oxide (Al₂O₃), hafnium oxide (HfO₂), silicon nitride (Si₃N₄), or silicon oxide (SiO₂), wherein the surface is present on a plurality of micropillars.

24. The vessel of claim 23, wherein the plurality of micropillars have at least one of the following characteristics: i) a micropillar height of from approximately 190-200 μm; ii) a micropillar width of approximately 20 um; a center-to-center micropillar distance of approximately 50 μm; iii) an interpillar distance of about 30 μm.

25. The vessel of claim 23, wherein the vessel is present in a microfluidic device.

26. A kit comprising the device of claim 25, the kit further comprising at least one buffer for use in adhering polynucleotides to the micropillars.

27. The kit of claim 26, further comprising at least one polymerase.

28. The kit of claim 26, further comprising oligonucleotide primers specific for a genomic sequence of one or more pathogenic microorganisms.

29. The kit of claim 28, further comprising a cartridge adapted to introduce a sample into a microfluidic vessel.

30. A process for detecting nucleic acids from a pathogen comprising:
a. contacting a biological sample from a pathogen comprising or suspected of comprising nucleic acids with a surface, wherein the nucleic acids if present adhere to the surface;
b. washing the adhered nucleic acids and the surface with a first buffer;
c. washing the nucleic acids and the surface with a second buffer, wherein the second buffer has a pH that is equal to or higher than the first buffer;
d. amplifying at least some of the nucleic acids to produce an amplification product; and
e. detecting the amplification product;
wherein the step of contacting the nucleic acids with the surface is performed using an adhesion solution comprising kosmotropic salts and optionally a nuclease inhibitor, and wherein the process takes less than one hour.

31. The process of claim 30, wherein the pathogen is HCV, HIV, Zika, or HPV.

32. The process of claim 30, wherein the process takes less than 25 minutes.

33. The process of claim 30, wherein the amplifying is conducted in a PCR chamber.

34. The process of claim 33, wherein the PCR chamber is a silicon microchannel.

35. The process of claim 34, wherein the silicon microchannel has one or more meanders.

36. The process of claim 35, wherein the silicon microchannel has nine meanders.

37. The process of claim 34, wherein the silicon microchannel has a volume of 1.3 μL.
